(19) United States
(12) Patent Application Publication
(10) Pub. No.: US 2009/0131483 A1
(43) Pub. Date: May 21, 2009

(54) 2-PYRIDINE DERIVATIVES AS INHIBITORS OF NEUTROPHILE ELASTASE

(76) Inventors: Peter Hansen, Lund (SE); Karolina Lawitz, Lund (SE); Hans Lonn, Lund (SE); Antonios Nikitidis, Lund (SE)

Correspondence Address: MORGAN LEWIS & BOCKIUS LLP, 1111 PENNSYLVANIA AVENUE NW, WASHINGTON, DC, 20004, US

(21) Appl. No.: 11/908,746

(22) PCT Filed: Mar. 14, 2006
(86) PCT No.: PCT/SE2006/000327

§ 371 Date: Oct. 7, 2008

(30) Foreign Application Priority Data
Mar. 16, 2005 (SE) 0500604-4

(51) Int. Cl. A61K 31/4439; C07D 413/14; C07D 413/02; A61P 29/00; A61P 11/00; A61K 31/44; C07D 213/81

(52) U.S. Cl. 514/341; 546/272.1; 546/323; 514/340; 514/354

(57) ABSTRACT

The invention provides compounds of formula (I) wherein R¹, R², R³, R⁴, R⁵ and X are as defined in the specification and optical isomers, racemates and tautomers thereof, and pharmaceutically acceptable salts thereof; together with processes for their preparation, pharmaceutical compositions containing them and their use in therapy. The compounds are inhibitors of human neutrophil elastase.

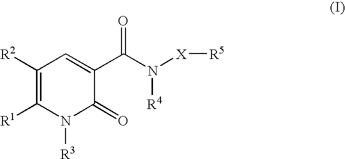

The present invention relates to 2-pyridone derivatives, processes for their preparation, pharmaceutical compositions containing them and their use in therapy.

Elastases are possibly the most destructive enzymes in the body, having the ability to degrade virtually all connective tissue components. The uncontrolled proteolytic degradation by elastases has been implicated in a number of pathological conditions. Human neutrophil elastase (hNE), a member of the chymotrypsin superfamily of serine proteases is a 33-KDa enzyme stored in the azurophilic granules of the neutrophils. In neutrophils the concentration of NE exceeded 5 mM and its total cellular amount has been estimated to be up to 3 pg. Upon activation, NE is rapidly released from the granules into the extacellular space with some portion remaining bound to neutrophil plasma membrane (See Kawabat et al. 2002, Eur. J. Pharmacol. 451, 1-10). The main intracellular physiological function of NE is degradation of foreign organic molecules phagocytosed by neutrophils, whereas the main target for extracellular clastase is elastin (Janoff and Scherer, 1968, J. Emp. Med. 128, 1137-1155). NE is unique, as compared to other proteases (for example, proteinase 3) in that it has the ability to degrade almost all extracellular matrix and key plasma proteins (See Kawabat et al., 2002, Eur. J. Pharmacol. 451, 1-10). It degrades a wide range of extracellular matrix proteins such as elastin, Type 3 and type 4 collagens, laminin, fibronectin, cytokines, etc. (Ohbayashi, H., 2002, Expert Opin. Investig. Drugs, 11, 965-980). NE is a major common mediator of many pathological changes seen in chronic lung disease including epithelial damage (Stockley, R. A. 1994, Am. 3. Resp. Crit. Care Med. 150, 109-113).

The destructive role of NE was solidified almost 40 years ago when Laurell and Eriksson reported an association of chronic airflow obstruction and emphysema with deficiency of serum α₁-antitrypsin (Laurell and Eriksson, 1963, Scand. J. Clin. Invest. 15, 132-140). Subsequently it was determined that α₁-antitrypsin is the most important endogenous inhibitor of human NE. The imbalance between human NE and endogenous antiprotease is believed to cause excess human NE in pulmonary tissues which is considered as a major pathogenic factor in chronic obstructive pulmonary disease (COPD). The excessive human NE shows a prominent destructive profile and actively takes part in destroying the normal pulmonary structures, followed by the irreversible enlargement of the respiratory airspaces, as seen mainly in emphysema. There is an increase in neutrophil recruitment into the lungs which is associated with increased lung elastase burden and emphysema in α₁-proteinase inhibitor-deficient mice (Cavarra et al., 1996, Lab. Invest. 75, 273-280). Individuals with higher levels of the NE-α₁ protease inhibitor complex in bronchioalveolar lavage fluid show significantly accelerated decline in lung functions compared to those with lower levels (Betsuyaku et al., 2000, Respiration, 67, 261-267). Instillation of human NE via the trachea in rats causes lung haemorrhage, neutrophil accumulation during acute phase and emphysematous changes during chronic phase (Karaki et al., 2002, Am. S. Resp. Crit. Care Med., 166, 496-500). Studies have shown that the acute phase of pulmonary emphysema and pulmonary haemorrhage caused by NB in hamsters can be inhibited by pre-treatment with inhibitors of NE (Fujie et al., 1999, Inflamm. Res. 48, 160-167).

Neutrophil-predominant airway inflammation and mucus obstruction of the airways are major pathologic features of COPD), including cystic fibrosis and chronic bronchitis. NE impairs mucin production, leading to mucus obstruction of the airways. NE is reported to increase the expression of major respiratory mucin gene, MUCSAC (Fischer, B. M & Voynow, 2002, Am. J. Respir. Cell Biol., 26, 447-452). Aerosol administration of NE to guinea pigs produces extensive epithelial damage within 20 minutes of contact (Suzuki et al., 1996, Am. J. Resp. Crit. Care Med., 153, 1405-1411). Furthermore NE reduces the ciliary beat frequency of human respiratory epithelium in vitro (Smallman et al., 1984, Thorax, 39, 663-667) which is consistent with the reduced mucociliary clearance that is seen in COPD patients (Currie et al., 1984, Thorax, 42, 126-130). The instillation of NE into the airways leads to mucus gland hyperplasia in hamsters (Lucey et al., 1985, Am. Resp. Crit. Care Med., 132, 362-366). A role for NE is also implicated in mucus hypersecretion in asthma. In an allergen sensitised guinea pig acute asthma model an inhibitor of NE prevented goblet cell degranulation and mucus hypersecretion (Nadel et al., 1999, Eur. Resp. J., 13, 190-196).

NE has been also shown to play a role in the pathogenesis of pulmonary fibrosis.

NE: α₁-protenase inhibitor complex is increased in serum of patients with pulmonary fibrosis, which correlates with the clinical parameters in these patients (Yamanouchi et al., 1998, Eur. Resp. J. 11, 120-125). In a murine model of human pulmonary fibrosis, a NE inhibitor reduced bleomycin-induced pulmonary fibrosis (Taooka et al., 1997, Am. J. Resp. Crit. Care Med., 156, 260-265). Furthermore investigators have shown that NE deficient mice are resistant to bleomycin-induced pulmonary fibrosis (Dunsmore et al., 2001, Chest, 120, 35S-36S). Plasma NE level was found to be elevated in patients who progressed to ARDS implicating the importance of NE in early ARDS disease pathogenesis. (Donnelly et al., 1995, Am. J. Res. Crit. Care Med., 151, 428-1433). The antiproteases and NE complexed with antiprotease are increased in lung cancer area (Marchandise et al., 1989, Eur. Resp. J. 2, 623-629). Recent studies have shown that polymorphism in the promoter region of the NE gene are associated with lung cancer development (Taniguchi et al, 2002, Clin. Cancer Res., 8, 1115-1120.

Acute lung injury caused by endotoxin in experimental animals is associated with elevated levels of NE (Kawabata, et al., 1999, Am. J. Resp. Crit. Care, 161, 2013-2018). Acute lung inflammation caused by intratracheal injection of lipopolysaccharide in mice has been shown to elevate the NE activity in bronchioalveolar lavage fluid which is significantly inhibited by a NE inhibitor (Fujie et al., 1999, Eur. J. Pharmacol., 374, 117-125; Yasui, et al., 1995, Eur. Resp. J., 8, 1293-1299). NE also plays an important role in the neutrophil-induced increase of pulmonary microvascular permeability observed in a model of acute lung injury caused by tumour necrosis factor α (TNFα) and phorbol myristate acetate (PMA) in isolated perfused rabbit lungs (Miyazaki et al., 1999, Am. J. Respir. Crit. Care Med., 157, 89-94).

A role for NE has also been suggested in monocrotoline-induced pulmonary vascular wall thickening and cardiac hypertrophy (Molteni et al., 1989, Biochemical Pharmacol. 38, 2411-2419). Serine elastase inhibitor reverses the monocrotaline-induced pulmonary hypertension and remodelling in rat pulmonary arteries (Cowan et al., 2000, Nature Medicine, 6, 698-702). Recent studies have shown that serine elastase, that is, NE or vascular elastase are important in cigarette smoke-induced muscularisation of small pulmonary arteries in guinea pigs (Wright et al., 2002, Am. J. Respir. Crit. Care Med., 166, 954-960).

NE plays a key role in experimental cerebral ischemic damage (Shimakura et al., 2000, Brain Research, 858, 55-60), ischemia-reperfusion lung injury (Kishima et al., 1998, Ann. Thorac. Surg. 65, 913-918) and myocardial ischemia in rat heart (Tiefenbacher et al., 1997, Eur. J. Physiol., 433, 563-570). Human NE levels in plasma are significantly increased above normal in inflammatory bowel diseases, for example, Crohn's disease and ulcerative colitis (Adeyemi et al., 1985, Gut, 26, 1306-1311). In addition NE has also been assumed to be involved in the pathogenesis of rheumatoid arthritis (Adeyemi et al., 1986, Rheumatol. Int., 6, 57). The development of collagen induced arthritis in mice is suppressed by a NE inhibitor (Kakimoto et al., 1995, Cellular Immunol. 165, 26-32).

Thus, human NE is known as one of the most destructive serine proteases and has been implicated in a variety of inflammatory diseases. The important endogenous inhibitor of human NE is α₁-antitrypsin. The imbalance between human NE and antiprotease is believed to give rise to an excess of human NE resulting in uncontrolled tissue destruction. The protease/antiprotease balance may be upset by a decreased availability of α₁-antitrypsin either through inactivation by oxidants such as cigarette smoke, or as a result of genetic inability to produce sufficient serum levels. Human NE has been implicated in the promotion or exacerbation of a number of diseases such as pulmonary emphysema, pulmonary fibrosis, adult respiratory distress syndrome (ARDS), ischemia reperfusion injury, rheumatoid arthritis and pulmonary hypertension.

In accordance with the present invention, there is therefore provided a compound of formula

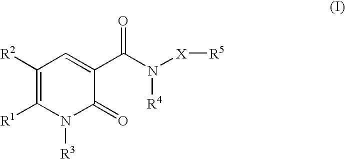

wherein
R¹ represents hydrogen or C₁-C₆ alkyl;
R² represents halogen, cyano, carboxyl, hydroxyl, nitro, —C(O)H, —C(O)NR¹⁰R¹¹, —NR¹²R¹³ or a group selected from C₁-C₆ alkyl, C₁-C₆ alkoxy, C₁-C₆ alkylcarbonyl, C₁-C₆ alkoxycarbonyl, C₂-C₆ alkenyl, C₂-C₆ alkynyl and a saturated or unsaturated 3- to 10-membered ring system optionally comprising at least one ring heteroatom selected from nitrogen, oxygen and sulphur, each group being optionally substituted by one or more substituents independently selected from halogen, cyano, carboxyl, hydroxyl, oxygen, nitro, —S(O)ₚR¹⁵, —NR¹⁶S(O)_{q}R¹⁷, —C(O)NR¹⁸R¹⁹, C₁-C₆ alkyl, C₁-C₆ alkoxy, C₁-C₆ alkylcarbonyl, C₁-C₆ alkoxycarbonyl and a saturated or unsaturated 5- to 6-membered monocyclic ring system optionally comprising at least one ring heteroatom selected from nitrogen, oxygen and sulphur;
R³ represents a phenyl group substituted with at least one substituent selected from halogen, cyano, nitro, trifluoromethyl or methylcarbonyl;
R⁴ represents hydrogen or C₁-C₆ alkyl optionally substituted with at least one is substituent selected from hydroxyl and C₁-C₆ alkoxy;
X represents a bond or a group —C₁-C₆ alkylene-Y—, wherein Y represents a single bond, oxygen atom, NR²⁴ or S(O)_{w};
R⁵ represents a monocyclic ring system selected from
i) phenoxy,
ii) phenyl,
iii) a 5- or 6-membered heteroaromatic ring comprising at least one ring heteroatom selected from nitrogen, oxygen and sulphur,
iv) a saturated or partially unsaturated C₃-C₆ hydrocarbyl ring, or
v) a saturated or partially unsaturated 4- to 7-membered heterocyclic ring comprising at least one ring heteroatom selected from oxygen, S(O)ᵣ and NR²⁰, wherein at least one of the ring carbon atoms may be optionally replaced by a carbonyl group;
   R⁵ being substituted by at least one substituent selected from oxygen, C₃-C₈ cycloalkyl, —S(O)ᵥR²¹, and C₁-C₆ alkyl substituted with at least one substituent selected from cyano, hydroxyl, C₁-C₆ alkoxy, C₁-C₆ alkylthio and —C(O)NR²²R²³;
   R¹⁰, R¹¹, R¹² and R¹³ each independently represent hydrogen or C₁-C₆ alkyl;
   p is 0, 1 or 2;
   q is 0, 1 or 2;
   r is 0, 1 or 2;
   w is 0, 1 or 2;
   R¹⁵, R¹⁶, R¹⁷, R¹⁸ and R¹⁹ each independently represent hydrogen or C₁-C₆ alkyl;
   R²⁰ represents hydrogen, C₁-C₆alkyl, C₁-C₆alkylcarbonyl or C₁-C₆ alkoxycarbonyl;
   v is 0, 1 or 2;
   R²¹ represents hydrogen, C₁-C₆ alkyl or C₃-C₈ cycloalkyl;
   R²² and R²³ each independently represent hydrogen or C₁-C₆ alkyl;
   R²⁴ represents hydrogen or C₁-C₆ alkyl;
      with the proviso that when R⁵ is substituted with a C₃-C₈ cycloalkyl or an —S(O)ᵥR²¹ substituent group, then R² represents either

(a) a substituted C₁-C₆ alkyl group in which at least one substituent group is cyano, carboxyl, —S(O)ₚR¹⁵, —NR¹⁶S(O)_{q}R¹⁷, —C(O)NR¹⁸R¹⁹ or C₁-C₆ alkoxycarbonyl,

(b) a substituted C₂-C₆ alkynyl group in which at least one substituent group is hydroxyl, or

(c) a substituted C₁-C₆ alkoxy group in which at least one substituent group is a 5- to 6-membered saturated or unsaturated monocyclic ring system optionally comprising at least one ring heteroatom selected from nitrogen, oxygen and sulphur;

or a pharmaceutically acceptable salt thereof.

In the context of the present specification, unless otherwise stated, an alkyl, alkenyl or alkynyl substituent group or an alkyl moiety in a substituent group may be linear or branched. Similarly, an alkylene group may be linear or branched. In the definition of R² the saturated or unsaturated 3- to 10-membered ring system and the saturated or unsaturated 5- to 6-membered monocyclic ring system may each have alicyclic or aromatic properties. An unsaturated ring system will be partially or fully unsaturated.

R¹ represents hydrogen or C₁-C₆ alkyl (e.g. methyl, ethyl, n-propyl, isopropyl, n-butyl, isobutyl, tert-butyl, n-pentyl or n-hexyl).

In one embodiment of the invention, R¹ represents a C₁-C₄ or C₁-C₂ alkyl group, in pasticular a methyl group.

R² represents halogen (e.g. fluorine, chlorine, bromine or iodine), cyano, carboxyl, hydroxyl, nitro, —C(O)H, C(O)NR¹⁰R¹¹, —NR¹²R¹³, or

a group selected from
C₁-C₆ alkyl (e.g. methyl, ethyl, n-propyl, isopropyl, n-butyl, isobutyl, tert-butyl, n-pentyl or n-hexyl),
C₁-C₆ alkoxy (e.g. methoxy, ethoxy, n-propoxy, isopropoxy, n-butoxy, isobutoxy, tert-butoxy, n-pentoxy or n-hexoxy),
C₁-C₆ alkylcarbonyl (e.g. methylcarbonyl, ethylcarbonyl, n-propylcarbonyl, isopropylcarbonyl, n-butylcarbonyl, isobutylcarbonyl, tert-butylcarbonyl, n-pentylcarbonyl or n-hexylcarbonyl),
C₁-C₆ alkoxycarbonyl (e.g. methoxycarbonyl, ethoxycarbonyl, n-propoxycarbonyl, isopropoxycarbonyl, n-butoxycarbonyl, isobutoxycarbonyl, tert-butoxycarbonyl, n-pentoxycarbonyl or n-hexoxycarbonyl),
C₂-C₆ alkenyl (e.g. ethenyl, prop-1-enyl, prop-2-enyl, but-1-enyl, pent-1-enyl, hex-1-enyl or 2-methyl-pent-2-enyl),
C₂-C₆ alkynyl (e.g. ethynyl, prop-1-ynyl, prop-2-ynyl, but-1ynyl, pent-1ynyl, hex-1ynyl or 2-methyl-pent-2-ynyl) and
a saturated or unsaturated 3- to 10-membered (e.g. 3-, 4- or 5- to 6-, 7-, 8-, 9- or 10-membered) ring system optionally comprising at least one ring heteroatom (e.g. one, two, three or four ring heteroatoms independently) selected from nitrogen, oxygen and sulphur,
   each group being optionally substituted by one or more (e.g. one, two, three or four) substituents independently selected from halogen (e.g. fluorine, chlorine, bromine or iodine), cyano, carboxyl, hydroxyl, oxygen, nitro, —S(O)ₚR¹⁵, —NR¹⁶S(O)_{q}R¹⁷, —C(O)NR¹⁸R¹⁹, C₁-C₆ alkyl (e.g. methyl, ethyl, n-propyl, isopropyl, n-butyl, isobutyl, tert-butyl, n-pentyl or n-hexyl), C₁-C₆ alkoxy (e.g. methoxy, ethoxy, n-propoxy, isopropoxy, n-butoxy, isobutoxy, tert-butoxy, n-pentoxy or n-hexoxy), C₁-C₆ alkylcarbonyl (e.g. methylcarbonyl, ethylcarbonyl, n-propylcarbonyl, isopropylcarbonyl, n-butylcarbonyl, isobutylcarbonyl, tert-butylcarbonyl, n-pentylcarbonyl or n-hexylcarbonyl), C₁-C₆ alkoxycarbonyl (e.g. methoxycarbonyl, ethoxycarbonyl, n-propoxycarbonyl, isopropoxycarbonyl, n-butoxycarbonyl, isobutoxycarbonyl, tert-butoxycarbonyl, n-pentoxycarbonyl or n-hexoxycarbonyl) and a saturated or unsaturated 5- to 6-membered monocyclic ring system optionally comprising at least one ring heteroatom (e.g. one, two, three or four ring heteroatoms independently) selected from nitrogen, oxygen and sulphur.

Examples of saturated or unsaturated 3- to 10-membered ing systems that may be used, which may be monocyclic or polycyclic (e.g. bicyclic) in which the two or more rings are fused, include one or more (in any combination) of cyclopropyl, cyclobutyl, cyclopentyl, cyclohexyl, bicyclo[2.2.1]heptyl, cyclopentenyl, cyclohexenyl, phenyl, pyrrolidinyl, piperidinyl, piperazinyl, morpholinyl, thiomorpholinyl, diazabicyclo[2.2.1]hept-2-yl, naphthyl, benzofuranyl, benzothienyl, benzodioxolyl, quinolinyl, oxazoyl, 2,3-dihydrobenzofuranyl, tetrahydropyranyl, pyrazolyl, pyrazinyl, thiazolidinyl, indanyl, thienyl, isoxazolyl, pyridazinyl, thiadiazolyl, pyrrolyl, furanyl, thiazolyl, indolyl, imidazolyl, pyrimidinyl, benzimidazolyl, triazolyl, tetrazolyl and pyridinyl.

Preferred ring systems include cyclopropyl, isoxazolyl and pyrazolyl.

Examples of saturated or unsaturated 5- to 6-membered monocyclic ring systems that may be used include pyrrolidinyl, piperazinyl, morpholinyl, furanyl, thienyl, pyrrolyl, phenyl, oxazolyl, 1,2,4-oxadiazolyl, 1,3,4-oxadiazolyl, isoxazolyl, imidazolyl, pyrazolyl, thiazolyl, triazolyl, thiadiazolyl, pyridinyl, pyridinyl, pyrazinyl and tetrazolyl. Preferred ring systems include morpholinyl and piperazinyl.

In an embodiment of the invention, R² represents halogen, cyano, carboxyl, hydroxyl, nitro, —C(O)H, —C(O)NR¹⁰R¹¹, —NR¹²R¹³, or a group selected from C₁-C₆, or C₁-C₄, alkyl, C₁-C₆, or C₁-C₄, alkoxy, C₁-C₆, or C₁-C₄, alkylcarbonyl, C₁-C₆, or C₁-C₄, alkoxycarbonyl, C₂-C₄ alkenyl, C₂-C₄ alkynyl and a saturated or unsaturated 3- to 6-membered ring system optionally comprising one or two ring heteroatoms independently selected from nitrogen, oxygen and sulphur, each group being optionally substituted by one or two substituents independently selected from halogen, cyano, carboxyl, hydroxyl, oxygen, nitro, —S(O)ₚR¹⁵, —NR¹⁶S(O)_{q}R¹⁷, —C(O)NR¹⁸R¹⁹, C₁-C₆, or C₁-C₄, alkyl, C₁-C₆, or C₁-C₄, alkoxy, C₁-C₆, or C₁-C₄, alkylcarbonyl, C₁-C₆, or C₁-C₄, alkoxycarbonyl and a saturated or unsaturated 5- to 6-membered monocyclic ring system optionally comprising one or two ring heteroatoms independently selected from nitrogen, oxygen and sulphur.

In another embodiment of the invention, R² represents halogen or a group selected from C₁-C₄ alkyl, C₁-C₄ alkoxy, C₂-C₄ alkynyl and a saturated or unsaturated 3- to 6-membered ring system optionally comprising two ring heteroatoms independently selected from nitrogen and oxygen, each group being optionally substituted by one or two substituents independently selected from cyano, carboxyl, hydroxyl, —S(O)ₚR¹⁵, —NR¹⁶S(O)_{q}R¹⁷, —C(O)NR¹⁸R¹⁹, C₁-C₄ alkyl, C₁-C₄ alkoxycarbonyl and a saturated or unsaturated 5- to 6-membered monocyclic ring system optionally comprising two ring heteroatoms independently selected from nitrogen and oxygen.

In a further embodiment of the invention, R² represents iodine or a group selected from methyl, ethyl, n-propyl, n-propoxy, prop-1-ynyl, cyclopropyl, isoxazolyl and pyrazolyl, each group being optionally substituted by one or two substituents independently selected from cyano, carboxyl, hydroxyl, —S(O)ₚR¹⁵, —NR¹⁶S(O)_{q}R¹⁷, —C(O)NR¹⁸R¹⁹, methyl, ethoxycarbonyl and morpholinyl.

R³ represents a phenyl group substituted with at least one substituent (e.g. one, two or three substituents independently) selected from halogen (e.g. fluorine, chlorine, bromine or iodine), cyano, nitro, trifluoromethyl or methylcarbonyl.

In one embodiment, R³ represents a phenyl group substituted with one or two substituents independently selected from fluorine, chlorine, cyano, nitro, trifluoromethyl or methylcarbonyl.

In another embodiment, R³ represents a phenyl group substituted with one substituent selected from fluorine, chlorine or trifluoromethyl.

In still another embodiment, R³ represents a phenyl group substituted with a trifluoromethyl substituent (preferably in the meta position).

R⁴ represents hydrogen or C₁-C₆ alkyl (e.g. methyl, ethyl, n-propyl, isopropyl, n-butyl, isobutyl, tert-butyl, n-pentyl or n-hexyl) optionally substituted with at least one substituent (e.g. one or two substituents independently) selected from hydroxyl and C₁-C₆ alkoxy (e.g. methoxy, ethoxy, n-propoxy, isopropoxy, n-butoxy, isobutoxy, tert-butoxy, n-pentoxy or n-hexoxy).

In one embodiment, R⁴ represents hydrogen or C₁-C₄ alkyl optionally substituted with one or two substituents independently selected from hydroxyl and C₁-C₄ alkoxy.

In another embodiment, R⁴ represents hydrogen.

X represents a bond or a group —C₁-C₆ alkylene-Y—. For the avoidance of doubt, X is orientated such that Y is attached to R⁵ in formula (I).

In an embodiment of the invention, Y represents a single bond and the alkylene moiety is a linear C₁-C₆, or C₁-C₄, alkylene.

In another embodiment of the invention, X represents methylene.

R⁵ represents a monocyclic ring system selected from
i) phenoxy,
ii) phenyl,
iii) a 5- or 6-membered heteroaromatic ring comprising at least one ring heteroatom (e.g. one, two, three or four ring heteroatoms independently) selected from nitrogen, oxygen and sulphur,
iv) a saturated or partially unsaturated C₃-C₆ hydrocarbyl ring, or
v) a saturated or partially unsaturated 4- to 7-membered heterocyclic ring comprising at least one ring heteroatom (e.g. one, two, three or four ring heteroatoms independently) selected from oxygen, S(O)ᵣ and NR²⁰, wherein at least one of the ring carbon atoms may be optionally replaced by a carbonyl group;
   the monocyclic ring system being substituted (on a ring atom) by at least one substituent (e.g. one, two or three substituents independently) selected from oxygen (e.g. to form an N-oxide), C₃-C₈ cycloalkyl (cyclopropyl, cyclobutyl, cyclopentyl, cyclohexyl, cycloheptyl or cyclooctyl), —S(O)ᵥR²¹, and C₁-C₆ alkyl (e.g. methyl, ethyl, n-propyl, isopropyl, n-butyl, isobutyl, tert-butyl, n-pentyl or n-hexyl) substituted with at least one substituent (e.g. one, two or three substituents independently) selected from cyano, hydroxyl, C₁-C₆ alkoxy (e.g. methoxy, ethoxy, n-propoxy, isopropoxy, n-butoxy, isobutoxy, tert-butoxy, n-pentoxy or n-hexoxy), C₁-C₆ alkylthio (e.g. methylthio, ethylthio, n-propylthio, isopropylthio, n-butylthio, isobutylthio, tert-butylthio, n-pentylthio or n-hexylthio) and —C(O)NR²²R²³.

Examples of a 5- or 6-membered heteroaromatic ring include furanyl, thienyl, pyrrolyl, oxazolyl, 1,2,4-oxadiazolyl, 1,3,4-oxadiazolyl, isoxazolyl, imidazolyl, pyrazolyl, thiazolyl, triazolyl, tetrazolyl, thiadiazolyl, pyridinyl, pyrimidinyl and pyrazinyl. Preferred heteroaromatic rings include isoxazolyl.

Unless otherwise indicated, a “saturated or partially unsaturated C₃-C₆ hydrocarbyl ring” denotes a 3- to 6-membered non-aromatic hydrocarbyl ring optionally incorporating one or more double bonds, examples of which include cyclopropyl, cyclobutyl, cyclopentyl, cyclohexyl, cyclopentenyl and cyclohexenyl.

Unless otherwise indicated, a “saturated or partially unsaturated 4- to 7-membered heterocyclic ring” as specified above denotes a 4- to 7-membered non-aromatic heterocyclic ring optionally incorporating one or more double bonds and optionally incorporating a carbonyl group, examples of which include tetrahydrofuranyl, tetramethylene sulfonyl, tetrahydropyranyl, 4-oxo-4H-pyranyl (4H-pyran-4-onyl), pyrrolidinyl, 3-pyrrolinyl, imidazolidinyl, 1,3-dioxolanyl (1,3-dioxacyclopentanyl), piperidinyl, piperazinyl, morpholinyl, perhydroazepinyl (hexamethylene iminyl), pyrrolidonyl and piperidonyl.

In a further embodiment of the invention, R⁵ represents a monocyclic ring system selected from
i) phenoxy,
ii) phenyl,
iii) a 5- or 6-membered heteroaromatic ring comprising one or two ring heteroatoms independently selected from nitrogen, oxygen and sulphur,
iv) a saturated or partially unsaturated C₃-C₆ hydrocarbyl ring, or
v) a saturated or partially unsaturated 4- to 7-membered heterocyclic ring comprising one or two ring heteroatoms independently selected from oxygen, S(O)ᵣ and NR²⁰, wherein at least one of the ring carbon atoms may be optionally replaced by a carbonyl group;
   the monocyclic ring system being substituted by one or two substituents independently selected from C₃-C₆ cycloalkyl, —S(O)ᵥR²¹, and C₁-C₄ alkyl substituted with one or two substituents independently selected from cyano, hydroxyl, C₁-C₄ alkoxy, C₁-C₄ alkylthio and —C(O)NR²²R²³.

In a still further embodiment of the invention, R⁵ represents a monocyclic ring system selected from phenyl or a 5- or 6-membered heteroaromatic ring comprising one or two ring heteroatoms independently selected from nitrogen and oxygen, the monocyclic ring system being substituted by one or two substituents independently selected from C₃-C₆ cycloalkyl, —S(O)ᵥR²¹, and C₁-C₄ alkyl substituted with one or two substituents independently selected from cyano, hydroxyl, C₁-C₄ alkoxy, C₁-C₄ alkylthio and —C(O)NR²²R²³.

In another embodiment, R⁵ represents a monocyclic ring system selected from phenyl or a 5- or 6-membered heteroaromatic ring comprising one or two ring heteroatoms independently selected from nitrogen and oxygen (such as isoxazolyl), the monocyclic ring system being substituted by one or two substituents independently selected from cyclopropyl, —S(O)ᵥR²¹, methyl, ethyl and n-propyl, the alkyl groups in turn being substituted with one or two substituents independently selected from cyano, hydroxyl, methoxy, methylthio and —C(O)NR²²R²³.

In still another embodiment, R⁵ represents a monocyclic ring system selected from phenyl or a 5-membered heteroaromatic ring comprising two ring heteroatoms independently selected from nitrogen and oxygen, the monocyclic ring system being substituted by one substituent selected from cyclopropyl, —S(O)ᵥR²¹, methyl, ethyl and n-propyl, the alkyl groups in turn being substituted with one substituent selected from cyano, hydroxyl, methoxy, methylthio and —C(O)NR²²R²³.

R¹⁰, R¹¹, R¹² and R¹³ each independently represent hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R¹⁰, R¹¹, R¹² and R¹³ each independently represent hydrogen or methyl.

In one embodiment p is 2.

In one embodiment, q is 2.

R¹⁵, R¹⁶, R¹⁷, R¹⁸ and R¹⁹ each independently represent hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R¹⁵, R¹⁶, R¹⁷, R¹⁸ and R¹⁹ each independently represent hydrogen or methyl.

R²⁰ represents hydrogen, C₁-C₆ alkyl (e.g. methyl, ethyl, n-propyl, isopropyl, n-butyl, isobutyl, tert-butyl, n-pentyl or n-hexyl), C₁-C₆ alkylcarbonyl (e.g. methylcarbonyl (acetyl), ethylcarbonyl, n-propylcarbonyl, isopropylcarbonyl, n-butylcarbonyl, isobutylcarbonyl, tert-butylcarbonyl, n-pentylcarbonyl or n-hexylcarbonyl), or C₁-C₆ alkoxycarbonyl (e.g. methoxycarbonyl, ethoxycarbonyl, n-propoxycarbonyl, isopropoxycarbonyl, n-butoxycarbonyl, isobutoxycarbonyl, tert-butoxycarbonyl, n-pentoxycarbonyl or n-hexoxycarbonyl).

In an embodiment of the invention, R²⁰ represents hydrogen, C₁-C₄ alkyl, C₁-C₄ alkylcarbonyl or C₁-C₄ alkoxycarbonyl.

In a further embodiment, R²⁰ represents hydrogen, methyl, ethyl, methylcarbonyl, ethylcarbonyl, methoxycarbonyl or ethoxycarbonyl.

In one embodiment, v is 2.

R²¹ represents hydrogen, C₁-C₆ alkyl (e.g. methyl, ethyl, n-propyl, isopropyl, n-butyl, isobutyl, tert-butyl, n-pentyl or n-hexyl) or C₃-C₈ cycloalkyl (cyclopropyl, cyclobutyl, cyclopentyl, cyclohexyl, cycloheptyl or cyclooctyl).

In an embodiment according to the invention, R²¹ represents hydrogen, C₁-C₃ alkyl or C₃-C₆ cycloalkyl.

In another embodiment, R²¹ represents C₁-C₃ alkyl (particularly methyl or isopropyl) or cyclopropyl.

R²², and R²³ each independently represent hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R²² and R²³ each independently represent hydrogen.

R²⁴ represents hydrogen or C₁-C₆ alkyl (e.g. methyl, ethyl, n-propyl, isopropyl, n-butyl, isobutyl, tert-butyl, n-pentyl or n-hexyl).

In an embodiment of the invention, R²⁴ represents hydrogen.

In an embodiment of the invention,
R¹ represents methyl;
R² represents iodine or a group selected from methyl, ethyl, n-propyl, n-propoxy, prop-1-ynyl, cyclopropyl, isoxazolyl and pyrazolyl, each group being optionally substituted by one or two substituents independently selected from cyano, carboxyl, hydroxyl, —S(O)ₚR¹⁵, —NR¹⁶S(O)_{q}R¹⁷, —C(O)NR¹⁸R¹⁹, methyl, ethoxycarbonyl and to morpholinyl;
R³ represents a phenyl group substituted with a trifluoromethyl substituent;
R⁴ represents hydrogen;
X represents methylene;
R⁵ represents a monocyclic ring system selected from phenyl or a 5-membered heteroaromatic ring comprising two ring heteroatoms independently selected from nitrogen and oxygen, the monocyclic ring system being substituted by one substituent selected from cyclopropyl, —S(O)ᵥR²¹, methyl, ethyl and n-propyl, the alkyl groups in turn being substituted with one substituent selected from cyano, hydroxyl, methoxy, methylthio and —C(O)NR²²R²³.
p is 2;
q is 2;
R¹⁵, R¹⁶, R¹⁷, R¹⁸ and R¹⁹ each independently represent hydrogen or methyl;
v is 2;
R²¹ represents C₁-C₃ alkyl or cyclopropyl; and
R²² and R²³ each independently represent hydrogen.

Examples of compounds of the invention include:
N-{[3-(2-Hydroxyethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
5-(3,5-Dimethylisoxazol-4-yl)-N-{[3-(2-hydroxyethyl)isoxazol-5-yl]methyl}-6-methyl-2-oxo-1-[3-(trifluoromethyl)phenyl]-1,2-dihydropyridine-3-carboxamide;
N-{[3-(2-Hydroxyethyl)isoxazol-5-yl]methyl}-5-iodo-6-methyl-2-oxo-1-[3-(trifluoromethyl)phenyl]-1,2-dihydropyridine-3-carboxamide;
N-{[3-(Hydroxymethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
5-(3,5-Dimethylisoxazol-4-yl)-N-{[3-(hydroxymethyl)isoxazol-5-yl]methyl}-6-methyl-2-oxo-1-[3-(trifluoromethyl)phenyl]-1,2-dihydropyridine-3-carboxamide;
5-Ethyl-N-{[3-(hydroxymethyl)isoxazol-5-yl]methyl}-6-methyl-2-oxo-1-[3-(trifluoromethyl)phenyl]-1,2-dihydropyridine-3-carboxamide;
5-Cyclopropyl-N-{[3-(hydroxymethyl)isoxazol-5-yl]methyl}-6-methyl-2-oxo-1-[3-(trifluoromethyl)phenyl]-1,2-dihydropyridine-3-carboxamide;
N-{[3-(Methoxymethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
5-(3,5-Dimethylisoxazol-4-yl)-6-methyl-N-({3-[(methylthio)methyl]isoxazol-5-yl}methyl)-2-oxo-1-[3-(trifluoromethyl)phenyl]-1,2-dihydropyridine-3-carboxamide;
N-{[3-(3-Amino-3-oxopropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
N-{[3-(2-Cyanoethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
N-{[3-(3-Hydroxypropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
5-(3-Amino-3-oxopropyl)-N-[(3-cyclopropylisoxazol-5-yl)methyl]-6-methyl-2-oxo-1-[3-(trifluoromethyl)phenyl]-1,2-dihydropyridine-3-carboxamide;
5-(2-Cyanoethyl)-N-[(3-cyclopropylisoxazol-5-yl)methyl]-6-methyl-2-oxo-1-[3-(trifluoromethyl)phenyl]-1,2-dihydropyridine-3-carboxamide;
N-[(3-Cyclopropylisoxazol-5-yl)methyl]-5-[3-(dimethylamino)-3-oxopropyl]-6-methyl-2-oxo-1-[3-(trifluoromethyl)phenyl]-1,2-dihydropyridine-3-carboxamide;
3-{5-({[(3-Cyclopropylisoxazol-5-yl)methyl]amino}carbonyl)-2-methyl-6-oxo-1-[3-(trifluoromethyl)phenyl]-1,6-dihydropyridin-3-yl}propanoic acid;
N-[(3-Cyclopropylisoxazol-5-yl)methyl]-6-methyl-5-[3-(methylsulfonyl)propyl]-2-oxo-1-[3-(trifluoromethyl)phenyl]-1,2-dihydropyridine-3-carboxamide;
N-[(3-Cyclopropylisoxazol-5-yl)methyl]-6-methyl-5-{3-[(methylsulfonyl)amino]propyl}-2-oxo-1-[3-(trifluoromethyl)phenyl]-1,2-dihydropyridine-3-carboxamide;
6-Methyl-5-{3-[(methylsulfonyl)amino]propyl}-N-[4-(methylsulfonyl)benzyl]-2-oxo-1-[3-(trifluoromethyl)phenyl]-1,2-dihydropyridine-3-carboxamide;
5-(3-Hydroxyprop-1-yn-1-yl)-6-methyl-N-[4-(methylsulfonyl)benzyl]-2-oxo-1-[3-(trifluoromethyl)phenyl]-1,2-dihydropyridine-3-carboxamide;
5-(3-Amino-3-oxopropyl)-N-[4-(cyclopropylsulfonyl)benzyl]-6-methyl-2-oxo-1-[3-(trifluoromethyl)phenyl]-1,2-dihydropyridine-3-carboxamide;
N-[4-(Isopropylsulfonyl)benzyl]-6-methyl-5-(2-morpholin-4-ylethoxy)-2-oxo-1 [3-(trifluoromethyl)phenyl]-1,2-dihydropyridine-3-carboxamide;
N-[4-(Cyclopropylsulfonyl)benzyl]-6-methyl-5-[(methylsulfonyl)methyl]-2-oxo-1-[3-(trifluoromethyl)phenyl]-1,2-dihydropyridine-3-carboxamide;
5-(1-Cyanoethyl)-6-methyl-N-[4-(methylsulfonyl)benzyl]-2-oxo-1-[3-(trifluoromethyl)phenyl]-1,2-dihydropyridine-3-carboxamide;
Ethyl 3-{5-({[4-(cyclopropylsulfonyl)benzyl]amino}carbonyl)-2-methyl-6-oxo-1-[3-(trifluoromethyl)phenyl]-1,6-dihydropyridin-3-yl}propanoate;
3-{5-({[4-(Cyclopropylsulfonyl)benzyl]amino}carbonyl)-2-methyl-6-oxo-1-[3-(trifluoromethyl)phenyl]-1,6-dihydropyridin-3-yl}propanoic acid;
   and pharmaceutically acceptable salts of any one thereof.

The present invention further provides a process for the preparation of a compound of formula (I) or a pharmaceutically acceptable salt thereof as defined above which comprises,

(a) reacting a compound of formula

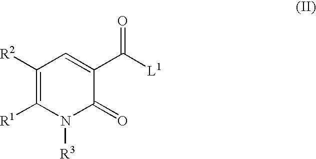

wherein L¹ represents a leaving group (such as halogen or hydroxyl) and R¹, R² and R³ are as defined in formula (a),
with a compound of formula

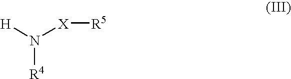

wherein X, R⁴ and R⁵ are as defined in formula (I); or
(b) when R² represents a halogen atom, reacting a compound of formula

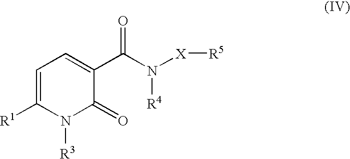

wherein X, R¹, R³, R⁴ and R⁵ are as defined in formula (I), with a halogenating agent

(such as N-iodosuccinimide in the presence of an acid such as trifluoroacetic acid or trifluoromethanesulphonic acid); or

(c) when R² is other than a halogen atom, reacting a compound of formula

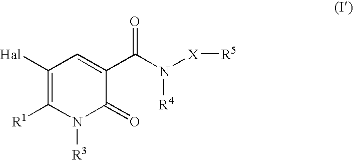

wherein Hal represents a halogen atom and X, R¹, R³, R⁴ and R⁵ are as defined in formula (I), with a nucleophile R^{2′}-M wherein R^{2′} is as defined in formula (I) other than a halogen atom and M represents an organo-tin or organo boronic acid group;
and optionally after (a), (b) or (c) carrying out one or more of the following:
converting the compound obtained to a further compound of the invention
forming a pharmaceutically acceptable salt of the compound.

In process (a), the reaction may conveniently be carried out in an organic solvent such as dichloromethane or N-methylpyrrolidinone at a temperature, for example, in the range from 0° C. to the boiling point of the solvent. If necessary or desired, a base and/or a coupling reagent such as HATU (O-(7-Azabenzotriazol-1-yl)-N,N,N′,N′-tetramethyluronium hexafluorophosphate), HOAT (1-Hydroxy-7-azabenzotriazole), HOBT (1-Hydroxybenzotriazole hydrate) or DIEA (N,N-Diisopropylethylamine) may be added.

In process (b), the reaction may conveniently be carried out in an organic solvent such as acetonitrile at a temperature, for example, in the range from 0° C. to 50° C. and in the presence of an acid such as trifluoromethanesulphonic acid.

In process (c), the reaction may conveniently be carried out in an organic solvent such as toluene at elevated temperature (i.e. above ambient temperature, 20° C.), for example, in the range from 50° C. to 150° C. and in the presence of a transition metal catalyst such as palladium. If necessary or desired, a base such as potassium carbonate may be added.

Compounds of formula (II) may be prepared by processes analogous to those described in (b) and (c) above, starting from compounds of formulae

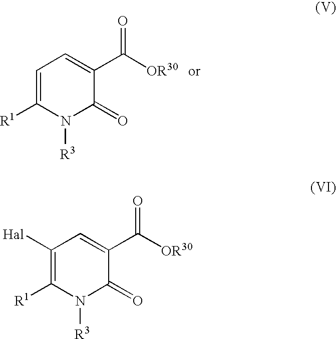

in which Hal represents a halogen atom, 100 represents hydrogen or C₁-C₆ alkyl and R¹ and R³ are as defined in formula (I).

Compounds of formula (IV) may be prepared by reacting a compound of formula

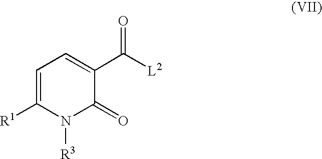

wherein L² represents a leaving group (such as halogen or hydroxyl) and R¹ and R³ are as defined in formula (I), with a compound of formula (III) as defined above under the same conditions as described above for process (a).

Compounds of formula (VII) are either commercially available, are known in the literature or may be prepared using known techniques. For example, compounds of formula (VII) in which L² represents a hydroxyl group may be prepared by condensing a compound of formula

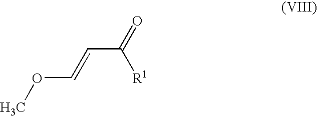

wherein R¹ is as defined in formula (I), with a compound of formula

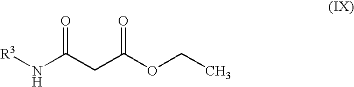

wherein R³ is as defined in formula (I), in the presence of a base (such as sodium methoxide), in an organic solvent (such as ethanol), followed by hydrolysis using a base such as sodium hydroxide.

Compounds of formulae (VIII) and (IX) are either known or may be prepared using methods that will be readily apparent to the man skilled in the art. For example, compounds of formula (VII can be prepared according to the methods of S. M Brombridge et al., Synthetic Communications, 1993, 23, 487-494 and compounds of formula (IX) can be prepared according to the methods of Igor V. Ukrainets et al., Tetrahedron, 1994, 50, 10331-10338.

Alternatively, compounds of formula (VII) in which L² represents a hydroxyl group and R¹ represents hydrogen may be prepared by reacting a compound of formula

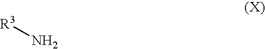

wherein R³ is as defined in formula (I), with a compound of formula

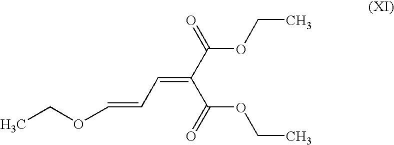

at a temperature of, for example, 160° C., followed by base promoted cyclisation and acid hydrolysis. The compound of formula (XI) can be prepared according to the disclosure of U.S. Pat. No. 3,838,155.

As another alternative, compounds of formula (VII) in which L² represents a hydroxyl group and R¹ represents methyl may be prepared by reacting a compound of formula

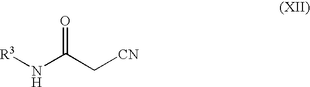

wherein R³ is as defined in formula (I), with 4-methoxy-3-buten-2-one. The reaction is conveniently carried out in an organic solvent such as diethyleneglycol monomethyl ether at a temperature, for example, of 20° C. to 110° C. and in the presence of a base such as 1,4-diazabicyclo[2.2.2]octane, followed by acid hydrolysis.

Compounds of formula (III), (V), (X) and (XII) are either commercially available, are known in the literature or may be prepared using known techniques.

Compounds of formula (I) can be converted into filer compounds of formula (I) using to standard procedures.

For example, compounds of formula (I) in which R² represents —C(O)NR¹⁰R¹¹ may be prepared by converting the corresponding carboxylic acid to the corresponding acyl chloride (i.e. in which the R² position is occupied by the substituent —C(O)Cl) which is then reacted with an amine of formula HNR¹⁰R¹¹ where R¹⁰ and R¹¹ are as defined above; or compounds of formula (I) in which R² represents —NR¹²R¹³ may be prepared by converting the corresponding carboxylic acid to the corresponding acyl azide (i.e. in which the R² position is occupied by the substituent —C(O)N₃) which is then reacted with an aqueous acid or a suitable (di)alkylamine in a solvent (e.g. toluene) at elevated temperature (e.g. in the range from 50° C. to 150° C.), the acid or (di)alkylamine being added after the acyl azide compound has been heated for a period of about 0.5 to 16 hours.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, sulphate, phosphate, acetate, fumarate, maleate, tartrate, lactate, citrate, pyruvate, succinate, oxalate, methanesulphonate or p-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) and their pharmaceutically acceptable salts have activity as pharmaceuticals, in particular as modulators of serine proteases such as proteinase 3 and pancreatic elastase and, especially, human neutrophil elastase, and may therefore be beneficial in the treatment or prophylaxis of inflammatory diseases and conditions.

Examples of such conditions include: adult respiratory distress syndrome (ARDS), cystic fibrosis, pulmonary emphysema, bronchitis, bronchiectasis, chronic obstructive pulmonary disease (COPD) and ischaemic-reperfusion injury. The compounds of this invention may also be useful in the modulation of endogenous and/or exogenous biological irritants which cause and/or propagate atherosclerosis, diabetes, myocardial infarction; hepatic disorders including but not limited to cirrhosis, systemic lupus erythematous, inflammatory disease of lymphoid origin, including but not limited to T lymphocytes, B lymphocytes, thymocytes; autoimmune diseases, bone marrow; inflammation of the joint (especially rheumatoid arthritis, osteoarthritis and gout); inflammation of the gastro-intestinal tract (especially inflammatory bowel disease, ulcerative colitis, pancreatitis and gastritis); inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); age related illness such as dementia, inflammatory diseases of cardiovascular origins; granulomatous diseases; renal diseases including but not limited to nephritis and polyarteritis; cancer; pulmonary hypertension, ingested poisons, skin contacts, stings, bites; asthma; rhinitis; HIV disease progression; for minimising the effects of organ rejection in organ transplantation including but not limited to human organs; and replacement therapy of proteinase inhibitors.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention also provides a method of treating, or reducing the risk of, a disease or condition in which inhibition of neutrophil elastase activity is beneficial which comprises administering to a patient in need thereof a therapeutically effective amount of a compound of formula (I) or a pharmaceutically acceptable salt thereof as hereinbefore defined.

The invention still further provides a method of treating, or reducing the risk of, an inflammatory disease or condition which comprises administering to a patient in need thereof a therapeutically effective amount of a compound of formula (I) or a pharmaceutically acceptable salt thereof as hereinbefore defined.

In particular, the compounds of this invention may be used in the treatment of adult respiratory distress syndrome (ARDS), cystic fibrosis, pulmonary emphysema, bronchitis, bronchiectasis, chronic obstructive pulmonary disease (COPD), pulmonary hypertension, asthma, rhinitis, ischemia-reperfusion injury, rheumatoid arthritis, osteoarthritis, cancer, atherosclerosis and gastric mucosal injury.

For the above-mentioned therapeutic uses the dosage administered will, of course, vary with the compound employed, the mode of administration, the treatment desired and the disorder indicated. The daily dosage of the compound of the invention may be in the range from 0.05 mg/kg to 100 mg/kg.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1999.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The present invention will now be further explained by reference to the following illustrative examples.

### General Methods

¹H NMR and ¹³C NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ₃ 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCF; Flow rate 0.7 ml/min; Wavelength 254 nm; Solvent A: water+0.1% TFA; Solvent B: acetonitrile+0.1% TFA; Gradient 15-95%/B 8 min, 95% B 1 min.

Analytical chromatography was run on a Symmetry Cl₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

The abbreviations or terms used in the examples have the following meanings:
HBTU: O-(Benzotriazol-1-yl)-N,N,N′,N′-tetramethyluronium hexafluorophosphate
HATU: O-(7-Azabenzotriazol-1-yl)-N,N,N′,N′-tetramethyluronium hexafluorophosphate
HOAT: 1-Hydroxy-7-azabenzotriazole
NMP: 1-N-Methyl-2-pyrrolidinone
DME: 1,2-Dimethoxyethane
THF: Tetrahydrofuran
TFA: Trifluoroacetic acid
DMF: N,N-Dimethylformamide
DCM/CH₂Cl₂: Dichloromethane
SOCl₂: Thionyl chloride
DIPEA: N,N-Diisopropylethylamine
EtOAc: Ethyl acetate
TEA: Triethylamine
NaOMe: Sodium methoxide
Pd: Palladium
Pd(PPh₃)₄: Palladium (0) tetrakistriphenylphosphine
Pd₂(DBA)₃: Tris(dibenzylideneacetone)dipalladium (0)
Pd(OAc)₂: Palladium (U) acetate
Pd(PPh₃)₂Cl₂: Palladium (V) triphenylphosphine chloride
MeOH: Methanol
p-TSA: p-Toluenesulphonic acid
Na₂CO₃: Sodium carbonate
NaOH: Sodium hydroxide
Na₂SO₄: Sodium sulphate
PPh₃: Triphenylphosphine
P(c-Hex)₃: Tricyclohexylphosphine
K₃PO₄: Potassium phosphate
TMS-polyphosphate: Tetramethylsilane-polyphosphate
NaHCO₃: Sodium hydrogencarbonate
CHCl₃: Trichloromethane
NCS: N-Chlorosuccinimide
KHCO₃: Potassium hydrogencarbonate
MeCN/CH₃CN: Acetonitrile
EtOH: Ethanol
CuI: Copper (I) Iodide
NaS₂O₄: Sodium hydrosulphite
DMSO: Dimethyl sulphoxide

### INTERMEDIATE EXAMPLE 1

### 6-Methyl-2-oxo-1-[3-(trifluoromethyl)phenyl]-1,2-dihydropyridine-3-carboxylic acid

### a) Ethyl 3-oxo-3-{[3-(trifluoromethyl)-phenyl]amino}propanoate

To an ice-cooled solution of 3-(trifluoromethyl)aniline (64.5 g, 0.40 mol) and triethylamine (60 ml) in acetone (700 ml) was added dropwise ethyl 3-chloro-3-oxopropanoate (63.6 g, 0.42 mol) in acetone (50 ml). After the addition (approx. 30 minutes) stirring was continued at room temperature overnight. The solvents were removed and water (1200 ml) was added. The resulting precipitate was filtered off, thoroughly washed twice with water and then dried to afford the sub-title compound as yellow powder (109 g, 99%). APCI-MS m/z: 276.1 [MH⁺].

### b) 6-Methyl-2-oxo-1-[3-(trifluoromethyl)phenyl]-1,2-dihydropyridine-3-carboxylic acid

To a solution of ethyl 3-oxo-3-{[3-(trifluoromethyl)phenyl]amino}propanoate (Example 1a, 19.2 g, 70 mmol) and sodium methoxide (7.6 g, 140 mmol) in EtOH (250 ml) was added 4-methoxybut-3-en-2-one (90%) (7.72 g, 77 mmol). After the addition, the reaction mixture was refluxed for 2 h and then cooled. Water (50 ml) and 2M NaOH were added and the mixture was stirred at room temperature overnight. The organic solvents were removed and the reaction mixture was extracted (washed) with EtOAc. The water phases were acidified with hydrochloric acid to pH 3-4, an orange coloured precipitate appeared and was filtered off; washed with water and dried. Recrystallisation twice from heptane/EtOAc (4:1) afforded the title compound (12 g, 580%) as a white powder.

¹H NMR (CDCl₃): δ 13.68 (1H, s); 8.54 (1H, d); 7.86 (1H, d); 7.79 (1H, t); 7.55 (1H, brs); 7.48 (1H, d); 6.58 (1H, d); 2.16 (3H, s).

APCI-MS m/z: 298.1 [MH⁺].

### EXAMPLE 1

### N-{[3-(2-Hydroxyethyl)isoxazol-5-yl]methyl}-6-methyl-5-(1-methyl-1-pyrazol-5-yl)-2-oxo-1-[3-(trifluoromethyl)phenyl]-1,2-dihydropyridine-3-carboxamide

### a) 5-Iodo-6-methyl-2-oxo-1-(3-trifluoromethyl-phenyl)-1,2-dihydro-pyridine-3-carboxylic acid prop-2-ynylamide

In a flask was dissolved 6-Methyl-2-oxo-1-(3-trifluoromethyl-phenyl)-1,2-dihydro-pyridine-3-carboxylic acid (prepared as described in Intermediate Example 1, 0.5 g, 1.68 mmol) in CH₂Cl₂ (7 ml) and TFA (3 ml). N-Iodosuccinimide (0.378 g, 1.68 mmol) was added and the mixture was stirred at room temperature for 1 hour. The volatiles were removed by evaporation giving the 5-iodinated product, which was dissolved in CH₂Cl₂ (5 ml) and SOCl₂ (5 ml) was added. The mixture was stirred for 1 hour, and was then concentrated thoroughly. The crude solid acid chloride was dissolved in 1,4-Dioxane (10 ml, dry), and mixture of Hunigs base (DIPEA, 1 ml) and Propargyl amine (0.165 g, 3 mmol) was added. The mixture was stirred vigorously for 5 minutes, evaporated, and partitioned between EtOAc and water. The organic phase was dried, and concentrated in vacuo. Purification on silica gave 0.46 g (580%) of the sub-title compound as a yellowish solid.

APCI-MS m/z: 461.2 [MH⁺].

### b) 5-Iodo-6-methyl-2-oxo-1-(3-trifluoromethyl-phenyl)-1,2-dihydro-pyridine-3-carboxylic acid {3-[2-(tetrahydro-pyran-2-yloxy-ethyl]-isoxazol-5-ylmethyl}-amide

5-Iodo-6-methyl-2-oxo-1-(3-trifluoromethyl-phenyl)-1,2-dihydro-pyridine-3-carboxylic acid prop-2-ynylamide (Example 1a, 0.1 g, 0.217 mmol), 2-(3-Nitro-propoxy)-tetrahydro-pyran (0.056 g, 0.3 mmol), Phenylisocyanate (0.071 g, 0.6 mmol) and TEA (Triethylamine, 2 drops), was dissolved in benzene (dry, 2.5 ml). The mixture was heated (90° C.) with stirring for 3 hours. Evaporation and purification on preparative HPLC followed by freeze-drying gave 0.063 g (46%) of a yellowish solid.

APCI-MS m/z: 548.2 [MH⁺].
c) N-{[3-(2-Hydroxyethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide

In a vial was dissolved 5-Iodo-6-methyl-2-oxo-1-(3-trifluoromethyl-phenyl)-1,2-dihydro-pyridine-3-carboxylic acid {3-[2-(tetrahydro-pyran-2-yloxy)-ethyl]-isoxazol-5-ylmethyl}-amide (Example 1b, 0.032 g, 0.051 mmol) and 1-Methyl-5-trimethylstannyl-1H-pyrazole (0.037 g, 0.152 mmol) in DME (Dimethoxy ethane, 2 ml). The solution was degassed with nitrogen and Pd(PPh₃)₄ (10 mg) was added. The vial was sealed and heated (120° C.) with stirring for 2 hours. The reaction mixture was filtered, and concentrated in vacuo. Dissolving the residue in MeOH (10 ml), adding p-TSA (p-Toluenesulfonic acid monohydrate, 10 mg), and stirring this mixture over night gave a crude mixture of the title compound. The mixture was evaporated in vacuo, purified on preparative HPLC and freeze-dried and giving 0.013 g (51%) of the title compound,

¹H NMR (DMSO-d₆): δ 9.83 (1H, t, J 6.3 Hz); 8.22 (1H, s); 8.02 (1H, s); 7.92 (1H, d, J 7.3 Hz); 7.87-7.80 (2H, m); 7.53 (1H, d, J 1.8 Hz); 6.33 (1H, d, J 1.8 Hz); 6.23 (1H, s); 4.74 (1H, t, J 5.0 Hz); 4.61 (2H, d, J 6.1 Hz); 3.72 (3H, s); 3.63 (2H, q, J 6.3 Hz); 2.69 (2H, t, J 6.3 Hz); 1.82 (3H, s)

APCI-MS m/z: 502.4 [MH⁺].

### EXAMPLE 2

### 5-(3,5-Dimethylisoxazol-4-yl)-N-{[3-(2-hydroxyethyl)isoxazol-5-yl]methyl}-6-methyl-2-oxo-1-[3-(trifluoromethyl)phenyl]-1,2-dihydropyridine-3-carboxamide

The compound was prepared according to the method described in Example 1c, starting from 5-Iodo-6-methyl-2-oxo-1-(3-trifluoromethyl-phenyl)-1,2-dihydro-pyridine-3-carboxylic acid {3-[2-(tetrahydro-pyran-2-yloxy)-ethyl]-isoxazol-5-ylmethyl}-amide (Example 1b, 0.032 g, 0.051 mmol), 3,5-Dimethylisoxazol-4-yl-boronic acid (0.020 g, 0.142 mmol) and Na₂CO₃ (2M, 1.5 ml), with the exception that the intermediate was partitioned between EtOAc/water and the organic phase purified on silica before the hydrolysis step. Purification on preparative HPLC and freeze-drying gave 0.010 g (38%) of the title compound as a white solid.

¹H NMR (DMSO-d₆): δ 9.88 (1H, t, J 6.3 Hz); 8.18 (1H, s); 8.01 (1H, d, J 5.5 Hz); 7.92 (1H, d, J 7.2 Hz); 7.87-7.79 (2H, m); 6.23 (1H, s); 4.60 (2H, d, J 6.3 Hz); 3.62 (2H, t, J 6.6 Hz); 2.69 (2H, t, J 6.6 Hz); 2.32-2.29 (3H, m) 2.14-2.10 (3H, m); 1.81 (3H, s)

APCI-MS m/z: 517.4 [MH⁺].

### EXAMPLE 3

### N-{[3-(2-Hydroxyethyl)isoxazol-5-yl]methyl}-5-iodo-6-methyl-2-oxo-1-[3-(trifluoromethyl)phenyl]-1,2-dihydropyridine-3-carboxamide

5-Iodo-6-methyl-2-oxo-1-(3-trifluoromethyl-phenyl)-1,2-dihydro-pyridine-3-carboxylic acid {3-[2-(tetrahydro-pyran-2-yloxy)-ethyl]-isoxazol-5-ylmethyl}-amide (Example 1b, 0.14 g, 0.22 mmol) was hydrolysed according to the hydrolysis step described in Example 1c. Purification on preparative HPLC and freeze-drying gave 0.060 g (50%) of the title compound as a white solid.

### INTERMEDIATE EXAMPLE 2

### 6-Methyl-5-(2-methyl-2H-pyrazol-3-yl)-2-oxo-1-(3-trifluoromethyl-phenyl)-1,2-dihydro-pyridine-3-carboxylic acid

### a) Ethyl 6-methyl-2-oxo-1-[3-(trifluoromethyl)phenyl]-1,2-dihydropyridine-3-carboxylate

A suspension of 6-methyl-2-oxo-1-[3-(trifluoromethyl)phenyl]-1,2-dihydropyridine-3-carboxylic acid (Intermediate Example 1b, 13.1 g, 43.9 mmol), sodium carbonate (5.2 g, 48.3 mmol) and iodoethane (10.6 g, 67.7 mmol) in NMP (60 ml) was stirred at ambient temperature for 19 hours under a nitrogen atmosphere. The reaction mixture was partitioned between ethyl acetate and water. The organic phase was collected, washed with water and brine, dried over sodium sulphate, filtered and concentrated in vacuo. The residue was purified by flash chromatography on silica eluting with tert-butyl methyl ether/methanol (10:0.4) to give the sub-title compound as a light brown solid (12.5 g, 87%).

¹H NMR (CDCl₃): δ 8.21 (1H, d, J 7.4 Hz); 7.75 (1H, d, J 7.8 Hz); 7.68 (1H, t, J 7.8 Hz); 7.49 (1H, s); 7.42 (1H, d, J 7.8 Hz); 6.25 (1H, d, J 7.4 Hz); 4.36 (2H, q, J 7.2 Hz); 2.03 (3H, s); 1.37 (3H, t, J 7.2 Hz).

APCI-MS m/z: 326.1 [MH⁺].

### b) Ethyl 5-iodo-6-methyl-2-oxo-1-[3-(trifluoromethyl)phenyl]-1,2-dihydropyridine-3-carboxylate

To a solution of ethyl 6-methyl-2-oxo-1-[3-(trifluoromethyl)phenyl]-1,2-dihydropyridine-3-carboxylate (Intermediate Example 2a, 9.9 g, 30.5 mmol) in CH₂Cl (45 ml) and TFA (38 ml) was added N-iodosuccinimide (6.89 g, 30.6 mmol) under a nitrogen atmosphere. After 19 h stirring at ambient temperature the solvent was concentrated in vacuo. To the residue were added ethyl acetate and saturated aqueous sodium hydrogencarbonate to neutralize the remaining TFA. The organic phase was collected, washed with water and brine, dried over sodium sulfate, filtered and concentrated in vacuo. The residue was sub-title compound as a yellow solid (11.4 g, 83%).

¹H NMR (CDCl₃): δ 8.52 (1H, s); 7.76 (1H, d, J 7.8 Hz); 7.69 (1H, t, J 7.9 Hz); 7.46 (1H, s); 7.38 (1H, d, J 7.7 Hz); 4.36 (2H, q, J 7.1 Hz); 2.26 (3H, s); 1.37 (3H, t, J 7.2 Hz).

APCI-MS m/z: 452.0 [MH⁺].

### c) 6-Methyl-5-(2-methyl-2H-pyrazol-3-yl)-2-oxo-1-(3-trifluoromethyl-phenyl)-1,2-dihydro-pyridine-3-carboxylic acid

Ethyl 5-iodo-6-methyl-2-oxo-1-[3-(trifluoromethyl)phenyl]-1,2-dihydropyridine-3-carboxylate (Intermediate Example 2b, 0.77 g, 1.7 mmol), DME (25 ml), 5-trimethylstannyl-1-methyl-1H-pyrazole (0.49 g, 2 mmol), Pd(PPh₃)₄ (0.10 g, 0.087 mmol) and a magnetic stirrer bar were placed in a pressure safe glass vessel. The vessel was sealed and heated (130° C.) with stirring overnight. LC-MS showed complete formation of the product. The mixture was allowed to cool, and was then diluted with EtOAc (50 ml), washed with water and brine, and further dried with Na₂SO₄. Filtration and evaporation and subsequent purification on silica gave the intermediate ester. This material was dissolved in THF (10 ml) and water (5 ml) and NaOH (2M, 1 ml, 2 mmol) was added. The mixture was stirred at 50° C. for 1 hour. The THF was evaporated off and the aqueous solution was acidified whereupon the product precipitated. The product was extracted with EtOAc. The extracts were dried (over Na₂SO₄) and evaporated to give the title compound (0.3 g, 47%) as a yellowish solid.

¹H NMR (DMSO-d₆): δ 13.80 (1H, s); 8.25 (1H, s); 8.07 (1H, s); 7.99-7.93 (1H, m); 7.90-7.85 (2H, m); 7.54 (1H, d, J 1.8 Hz); 6.36 (1H, d, J 1.8 Hz); 3.73 (3H, s); 1.86 (3H, s).

APCI-MS m/z: 363.3 [MH⁺].

### EXAMPLE 4

### N-{[3-(Hydroxymethyl)isoxazol-5-yl]methyl}-6-methyl-5-(1-methyl-1H-pyrazol-5-yl)-2-oxo-1-[3-(trifluoromethyl)phenyl]1,2-dihydropyridine-3-carboxamide

6-Methyl-5-(2-methyl-2H-pyrazol-3-yl)-2-oxo-1-(3-trifluoromethyl-phenyl)-1,2-dihydro-pyridine-3-carboxylic acid (Intermediate Example 2, 0.020 g, 0.053 mmol), HBTU (0.020 g, 0.053 mmol) and Hunigs base (DIPEA, 0.034 g, 0.26 mmol) was dissolved in 1,4-Dioxane (dry, 5 ml) and stirred at room temperature for 30 minutes. To this mixture was added 5-Aminomethyl-3-hydroxymethyl-isoxazole (0.014 g, 0.053 mmol). The obtained mixture was stirred over night, the solvent was evaporated, and the residue was purified on preparative HPLC. Freeze-drying gave 0.014 g (54%) of the title compound as a white solid.

¹H NMR (DMSO-d₆): δ 9.85 (1H, t, J 6.1 Hz); 8.21 (1H, s); 8.02 (1H, s); 7.92 (1H, d, J 7.3 Hz); 7.87-7.80 (2H, m); 7-53 (1H, d, J 1.8 Hz); 6.33 (1H, d, J 1.8 Hz); 6.27 (1H, s); 4.63 (2H, d, J 6.1 Hz); 4.44 (2H, s); 3.72 (3H, s); 1.82 (3H, s)

APCI-MS m/z: 488.2 [MH⁺].

### INTERMEDIATE EXAMPLE 3

### 5-(3,5-Dimethylisoxazol-4-yl)-6-methyl-2-oxo-1-[3-(trifluoromethyl)phenyl]-1,2-dihydropyridine-3-carboxylic acid

Ethyl 5-iodo-6-methyl-2-oxo-1-[3-(trifluoromethyl)phenyl]-1,2-dihydropyridine-3-carboxylate (Intermediate Example 2b, 72 g, 1.6 mmol), DME (20 ml), 3,5-dimethylisoxazolyl-4-boronic acid (0.28 g, 2 mmol), Pd₂(DBA)₃ (0.036 g, 0.039 mmol), PPh₃ (0.062 g, 0.23 mmol), 2M Na₂CO₃ (10 ml) and a magnetic stirrer bar were placed in a pressure safe glass vessel. The vessel was sealed and heated (120° C.) with stirring overnight. LC-MS showed complete formation of the required product (including hydrolysis of the ester). The mixture was allowed to cool, the aqueous phase was acidified, and the organic phase was diluted with EtOAc (50 ml) and the phases were allowed to separate. The organic phase was washed with water and brine, and further dried with Na₂SO₄. Filtration and evaporation gave a crude mixture which was purified by preparative HPLC giving the title compound (0.27 g, 43%) as a yellowish solid.

¹H NMR (DMSO-d₆): δ 13.93 (1H, s); 8.25 (1H, s); 8.07 (1H, s); 7.99-7.93 (1H, m); 7.89-7.85 (2H, m); 2.35 (3H, m); 2.15-2.10 (3H, m); 1.85 (3H, s).

APCI-MS m/z: 393.1 [MH⁺].

### EXAMPLE 5

### 5-(3,5-Dimethylisoxazol-4-yl)-N-{[3-(hydroxymethyl)isoxazol-5-yl]methyl}-6-methyl-2-oxo-1-[3-(trifluoromethyl)phenyl]-1,2-dihydropyridine-3-carboxamide

The compound was prepared according to the method described for Example 4, starting from 5-(3,5-dimethyl-isoxazol-4-yl)-6-methyl-2-oxo-1-(3-trifluoromethyl-phenyl)-1,2-dihydro-pyridine-3-carboxylic acid (Intermediate Example 3, 0.023 g, 0.059 mmol). Freeze-drying gave 0.019 g (64%) of the title compound as a white solid.

¹H NMR (DMSO-d₆): δ 9.89 (1H, t, J 6.1 Hz); 8.18 (1H, s); 8.01 (1H, d, J 5.3 Hz); 7.92 (1H, d, J 7.3 Hz); 7.87-7.80 (2H, m); 6.27 (1H, s); 5.41 (1H, t, J 6.0 Hz); 4.62 (2H, d, J 6.0 Hz); 4.44 (2H, d, J 5.7 Hz); 2.34 (311, ds); 2.13 (3H, ds); 1.81 (3H, s) APCI-MS m/z: 503.1 [MH⁺].

### INTERMEDIATE EXAMPLE 4

### 5-Ethyl-6-methyl-2-oxo-1-[3-(trifluoromethyl)phenyl]-1,2-dihydropyridine-3-carboxylic acid

### a) 5-Ethenyl-6-methyl-2-oxo-1-[3-trifluoromethyl)phenyl]-1,2-dihydropyridine-3-carboxylic acid ethyl ester

In a pressure safe glass vessel was added Ethyl 5-iodo-6-methyl-2-oxo-1-[3-(trifluoromethyl)phenyl]-1,2-dihydropyridine-3-carboxylate (Intermediate Example 2b, 1.0 g, 2.21 mmol), dimethoxyethane (DME, 30 ml), ethenyl-tributylstannane (1.4 g, 4.4 mmol), Pd(PPh₃)₄ (0.04 g, 0.035 mmol) and a magnetic stirrer. The vessel was sealed and heated (110° C.) with stirring for 5 hours. LC-MS showed complete formation of the intermediate ethyl ester. The mixture was allowed to cool and the solvent was evaporated in vacuo. The obtained oil was purified on silica (Heptane:EtOAc), giving 0.6 g (77%) of the desired intermediate as a slightly yellowish solid.

¹H NMR (DMSO): δ 8.32 (1H, s); 7.78 (1H, d, J 7.8 Hz); 7.83 (1H, s); 7.80 (1H, t, J 7.8 Hz); 7.66 (1H, d, J 7.8 Hz); 6.81 (1H, dd); 5.64 (1H, d, J 17.6 Hz); 5.28 (1H, d, J 11.2 Hz); 4.22 (2H, q, J 7.2 Hz); 2.00 (3H, s); 1.26 (31z, t, J 7.2 Hz)

APCI-MS m/z: 352.2 [MH⁺].

### b) 5-Ethyl-6-methyl-2-oxo-1-[3-(trifluoromethyl)phenyl]-1,2-dihydropyridine-3-carboxylic acid ethyl ester

In a flask was dissolved 5-Ethenyl-6-methyl-2-oxo-1-[3-(trifluoromethyl)phenyl]-1,2-dihydropyridine-3-carboxylic acid ethyl ester (Intermediate Example 4a, 0.6 g, 1.7 mmol) in EtOAc (20 ml) and EtOH (20 ml) and 5% Pd on charcoal (0.12 g). The mixture was hydrogenated at normal pressure and room temperature for 48 hours. The catalyst was removed by filtration through Celite®, and was subsequently concentrated in vacuo, giving 0.55 (91%) of the intermediate as a beige solid.

¹H NMR (DMSO): δ 8.04 (1H, s); 7.87 (1H, d, J 7.9 Hz); 7.81-7.76 (2H, m); 7.62 (1H, d, J 7.9 Hz); 4.20 (2H, q, J 7.1 Hz); 2.53-2.46 (2H, m); 1.92 (3H, s); 1.24 (311, t, J 7.1 Hz); 1.11 (3H, t, J 7.6 Hz)

APCI-MS ma/z: 354.1 [MH⁺].

### c) 5-Ethyl-6-methyl-2-oxo-1-[3-(trifluoromethyl)phenyl]-1,2-dihydropyridine-3-carboxylic acid

In a flask was dissolved 5-Ethyl-6-methyl-2-oxo-1-[3-(trifluoromethyl)phenyl]-1,2-dihydropyridine-3-carboxylic acid ethyl ester (Intermediate Example 4b, 0.55 g, 1.55 mmol) in THF (20 ml) and water (10 ml). To this stirred mixture was added NaOH (1M, 4 ml, 4 mmol), and the mixture was stirred for 3 hours at room temperature. LC-MS showed complete hydrolysis of the ester and THF was removed in vacuo. The aqueous solution was acidified and extracted with EtOAc (2×20 ml). The extracts were washed with water and brine, and finally dried over Na₂SO₄. Filtration and evaporation afforded 0.50 g (100%) of the title compound as a beige solid.

¹H NMR (DMSO): δ 14.25 (1H, s); 8.38 (1H, s); 7.97 (1H, s); 7.94 (1H, d, J 7.8 Hz); 7.85 (1H, t, J 7.7 Hz); 7.77 (1H, d, J 7.7 Hz); 2.62 (2H, q, J 7.5 Hz); 2.04 (3H, s); 1.15 (3H, t, J 7.5 Hz) APCI-MS m/z: 326.2 [MH⁺].

### EXAMPLE 6

### 5-Ethyl-N-{[3-(hydroxymethyl)isoxazol-5-yl]methyl}-6-methyl-2-oxo-1-[3-(trifluoromethyl)phenyl]-1,2-dihydropyridine-3-carboxamide

The compound was prepared according to the method described for Example 4, starting from 5-Ethyl-6-methyl-2-oxo-1-(3-trifluoromethyl-phenyl)-1,2-dihydro-pyridine-3-carboxylic acid (Intermediate Example 4, 0.032 g, 0.10 mmol). Freeze-drying gave 0.023 g (53%) of the title compound as a white solid.

¹H NMR (DMSO-d₆): δ 9.97 (1H, t, J 6.1 Hz); 8.33 (1H, s); 7.91-7.86 (2H, m); 7.81 (1H, t, J 7.9 Hz); 7.69 (1H, d, J 7.9 Hz); 6.25 (1H, s); 5.40 (1H, t, J 6.0 Hz); 4.61 (2H, d, J 6.0 Hz); 4.43 (2H, d, J 5.8 Hz); 2.57 (2H, q, J 7.4 Hz); 1.98 (3H, s); 1.14 (3H, t, J 7.5 Hz)

APCI-MS m/z: 436.5 [MH⁺].

### INTERMEDIATE EXAMPLE 5

### 5-Cyclopropyl-6-methyl-2-oxo-1-[3-(trifluoromethyl)phenyl]-1,2-dihydropyridine-3-carboxylic acid

Ethyl 5-iodo-6-methyl-2-oxo-1-[3-(trifluoromethyl)phenyl]-1,2-dihydropyridine-3-carboxylate (Intermediate Example 2b, 0.77 g, 1.6 mmol), toluene (35 ml), cyclopropyl-boronic acid (0.257 g, 3 mmol), Pd(OAc)_{z} (0.072 g, 0.35 mmol), P(c-Hex)₃ (0.169 g, 0.6 mmol), K₃PO₄ monohydrate (1.6 g, 6.9 mmol), water (0.7 ml) and a magnetic stirrer bar were placed in a pressure safe glass vessel. The vessel was sealed and heated (130° C.) with stirring overnight. LC-MS showed complete formation of the intermediate ethyl ester. The mixture was allowed to cool and the phases were diluted with EtOAc (50 ml) and water (50 ml) and the phases were allowed to separate. The organic phase was washed with water and brine, and further dried with Na₂SO₄. Filtration and evaporation gave a crude intermediate. This material was dissolved in THF (10 μl) and water (5 ml). To this solution was added NaOH (1M, 3 ml, 3 mmol) and the mixture was stirred for 2 h at 50° C. The THF was evaporated and the residual aqueous phase was acidified and extracted with EtOAc (2×20 ml). The extracts were washed with water and brine and finally dried over Na₂SO₄. Filtration and evaporation gave the title compound (0.19 g, 33%).

APCI-MS m/z: 338.1 [MH⁺].

### EXAMPLE 7

### 5-Cyclopropyl-N-{[3-(hydroxymethyl)isoxazol-5-yl]methyl}-6-methyl-2-oxo-1-[3-(trifluoromethyl)phenyl]-1,2-dihydropyridine-3-carboxamide

The compound was prepared according to the method described for Example 4, starting from 5-Cyclopropyl-6-methyl-2-oxo-1-[3-(trifluoromethyl)phenyl]-1,2-dihydro-pyridine-3-carboxylic acid (Intermediate Example 5, 0.030 g, 0.11 mmol, described before). Freeze-drying gave 0.021 g (47%) of the title compound as a white solid.

¹H NMR (DMSO-d₆): δ 9.94 (1H, t, J 6.0 Hz); 8.18 (1H, s); 7.92-7.86 (2H, m); 7.82 (1H, t, J 7.8 Hz); 7.70 (1H, d, J 7.8 Hz); 6.25 (1H, s); 5.40 (1H, t, J 6.0 Hz); 4.60 (2H, d, J 6.0 Hz); 4.43 (2H, d, J 6.0 Hz); 2.13 (3H, s); 1.98-1.79 (1H, m); 0.98-0.90 (2H, m); 0.64-0.57 (2H, m)

APCI-MS m/z: 448.4 [MH⁺].

### EXAMPLE 8

### N-{[3-(Methoxymethyl)isoxazol-5-yl]methyl}-6-methyl-5-(1-methyl-1H-pyrazol-5-yl)-2-oxo-4-[3-(trifluoromethyl)phenyl]-1,2-dihydropyridine-3-carboxamide

(3-Hydroxymethyl-isoxazol-5-ylmethyl)-carbamic acid tert-butyl ester (0.045 g, 0.2 mmol) was dissolved in 1,4-Dioxane (2 ml). Hunigs base (DIPEA, 0.050 g, 0.38 mmol) and Methanesulfonyl chloride (0.043 g, 0.30 mmol) was added. The mixture was allowed to stand for 1 hour at room temperature, and then concentrated in vacuo. The residue was dissolved in MeOH (2 ml), NaOMe (0.032 g, 0.6 mmol) was added in one portion and the mixture was heated (50° C.) with stirring over night. The mixture was concentrated in vacuo and dissolved in CH₂Cl₂ (5 ml) and TFA (1 ml). The mixture was allowed to stand for 1 hour, and concentrated again in vacuo yielding {[3-(methoxymethyl)isoxazol-5-yl]methyl}amine as crude product.

The title compound was prepared according to the method described for Example 4, starting from 6-Methyl-5-(2-methyl-2H-pyrazol-3-yl)-2-oxo-1-(3-trifluoromethyl-phenyl)-1,2-dihydro-pyridine-3-carboxylic acid (Intermediate Example 2, 0.038 g, 0.1 mmol) and using the crude product of {[3-(methoxymethyl)isoxazol-5-yl]methyl}amine described above as amine. Freeze-drying gave 0.010 g (20%) of the title compound as a white solid.

¹H NMR (DMSO-d₆): δ 9.86 (1H, t, J 6.0 Hz); 8.21 (1H, s); 8.02 (1H, s); 7.92 (1H, d, J 7.3 Hz); 7.87-7.80 (2H, m); 7.53 (1H, d, J 1.8 Hz); 6.33 (1H, d, J 1.8 Hz); 6.30 (1H, s); 4.63 (2H, d, J 6.1 Hz); 4.42 (2H, s); 3.71 (3H, s); 3.27 (3H, s); 1.82 (3H, s)

APCI-MS m/z: 502.6 [MH⁺].

### EXAMPLE 9

### 5-(3,5-Dimethylisoxazol-4-yl)-6-methyl-N-({3-[(methylthio)methyl]isoxazol-5-yl}methyl)-2-oxo-1-[3-(trifluoromethyl)phenyl]-1,2-dihydropyridine-3-carboxamide

5-(3,5-Dimethylisoxazol-4-yl)-N-{[3-hydroxymethylisoxazol-5-yl]methyl}-6-methyl-2-oxo-1-[3-(trifluoromethyl)phenyl]-1,2-dihydropyridine-3-carboxamide (Example 5, 0.050 g, 0.1 mmol) was dissolved in 1,4-Dioxane (3 ml), followed by the addition of Dimethyl disulfide (0.075 g, 0.8 mmol) and Triethylphosphine (0.094 g, 0.8 mmol). The mixture was heated (80° C.) under nitrogen atmosphere with stirring for 3 hours and then evaporated. The residue was purified by preparative HPLC and freeze-dried giving 0.040 g (75%) of the title compound as a white solid.

¹H NMR (DMSO-d₆): δ 9.89 (1H, t, J 6.1 Hz); 8.18 (1H, s); 8.01 (1H, d, J 5.3 Hz); 7.92 (1H, d, J 7.3 Hz); 7.87-7.80 (2H, m); 6.27 (1H, s); 4.62 (2H, d, J 6.1 Hz); 3.66 (2H, s); 2.31 (3H, m); 2.13 (3H, m); 1.99 (3H, s), 1.81 (3H, s)

APCI-MS m/z: 533.5 [MH⁺].

### EXAMPLE 10

### N-{[3-(3-Amino-3-oxopropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide

### a) 3-[5({[5-Iodo-6-methyl-2-oxo-1-(3-trifluoromethyl-phenyl)-1,2-dihydro-pyridine-3-carbonyl]-amino}-methyl)-isoxazol-3-yl]-propionic acid methyl ester

The compound was prepared according to the method described for Example 1b, starting from 5-Iodo-6-methyl-2-oxo-1-(3-trifluoromethyl-phenyl)-1,2-dihydro-pyridine-3-carboxylic acid prop-2-ynylamide (Example 1a) and methyl-4-nitrobutyrate. The crude product was purified on silica, eluting 0.14 g (47%) of material, pure enough for further synthesis.

APCI-MS m/z: 589.7 [MH⁺].

### b) N-{[3-(3-Amino-3-oxopropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide

3-[5({[5-Iodo-6-methyl-2-oxo-1-(3-trifluoromethyl-phenyl)-1,2-dihydro-pyridine-3-carbonyl]-amino}-methyl)-isoxazol-3-yl]-propionic acid methyl ester (Example 10a, 0.13 g, 0.22 mmol), was reacted with 1-Methyl-5-trimethylstannyl-1H-pyrazole (0.162 g, 6.6 mmol) according to the first part of the method described in Example 1c. The filtrate was dissolved in THF (10 ml). Water (2 ml) and NaOH (2M, 2 ml, 4 mmol) was added, and the solution was stirred for 1 hour at room temperature resulting in complete hydrolysis of the ester. TED was evaporated, the aqueous phase was diluted with water and acidified. Extraction with EtOAc, purification by preparative HPLC and freeze-drying gave 0.071 g (61%) of the intermediate carboxylic acid (6-Methyl-5-(2-methyl-2H-pyrazol-3-yl)-2-oxo-1-(3-trifluoromethyl-phenyl)-1,2-dihydro-pyridine-3-carboxylic acid [3-(2-carbamoyl-ethyl)-isoxazol-5-ylmethyl]-amide) as a white solid. This compound was dissolved in CH₂Cl₂ (5 ml), SOCl₂ (4 drops) was added, and the mixture was allowed to stand for 1 hour in a sealed flask. The mixture was concentrated in vacuo. The obtained acid chloride was dissolved in 1,4-Dioxane (dry, 5 ml), ammonia (32% aqueous, 2 ml) was added and the mixture was allowed to stand for 5 minutes at room temperature, followed by evaporation to dryness. The crude amide was purified by preparative HPLC, freeze-drying gave 0.039 g (33%) of the title compound as a white solid.

¹H NMR (DMSO-d₆): δ 9.83 (1H, t, J 6.0 Hz); 8.21 (1H, s); 8.02 (1H, s); 7.92 (1H, d, J 7.2 Hz); 7.87-7.80 (2H, m); 7.53 (1H, d, J 1.8 Hz); 7.32 (1H, bs); 6.80 (14, bs); 6.33 (1H, d, J 1.8 Hz); 6.18 (1H, s); 4.60 (2H, d, J 6.1 Hz); 3.72 (3H, s); 2.76 (2H, t, J 7.6 Hz); 2.38 (2H, t, J 7.6 Hz); 1.82 (3H, s)

APCI-MS m/z: 529.4 [MH⁺].

### EXAMPLE 11

### N-{[3-(2-Cyanoethyl)Isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide

N-{[3-(3-Amino-3-oxopropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide (Example 10, 0.022 g, 0.041 mmol) was dissolved in TMS-polyphosphate (2 ml, CH₂Cl₂-solution, Synthesis 1982 p 591-2) and heated (80° C.) with stirring for 2 hours. The mixture was diluted with CH₂Cl₂, washed with water and dried. Evaporation afforded a crude mixture of the compound, which was purified by preparative HPLC. Freeze-drying gave 0.016 g (75%) of the title compound as a white solid.

¹H NMR (DMSO-d₆): δ 9.85 (1H, t, J 6.1 Hz); 8.21 (1H, s); 8.02 (1H, s); 7.92 (1H, d, J 7.3 is Hz); 7.88-7.80 (2H, m); 7.53 (1H, d, J 1.8 Hz); 6.33 (1H, d, J 1.8 Hz); 6.32 (1H, s); 4.64 (2H, d, J 6.0 Hz); 3.72 (3H, s); 2.96-2.82 (4H, m); 1.82 (3H, s)

APCI-MS m/z: 511.3 [MH⁺].

### EXAMPLE 12

### N-{[3-(3-Hydroxypropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide

### a) Acetic acid 3-[5-({[5-iodo-6-methyl-2-oxo-1-(3-trifluoromethyl-phenyl)-1,2-dihydro-pyridine-3-carbonyl]-amino}1-methyl)-isoxazol-3-yl]-propyl ester

The compound was prepared according to the method described for Example 1b, starting from 5-Iodo-6-methyl-2-oxo-1-(3-trifluoromethyl-phenyl)-1,2-dihydro-pyridine-3-carboxylic acid prop-2-ynylamide (Example 1a) and 3-nitropropyl acetate. The crude product was purified on silica, giving 0.17 g (76%) of the sub-title compound.

APCI-MS m/z: 603.9 [MH⁺].

### b) N-{[3-(3-Hydroxypropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide

Acetic acid 3-[5-({[5-iodo-6-methyl-2-oxo-1-(3-trifluoromethyl-phenyl)-1,2-dihydro-pyridine-3-carbonyl]-amino}-methyl)-isoxazol-3-yl]-propyl ester (Example 12a, 0.16 g, 0.26 mmol), was reacted with 1-Methyl-5-trimethylstannyl-1H-pyrazole (0.162 g, 6.6 mmol) according to the first part of the method described in Example 1c. The solution was filtered, and purified on silica, giving 0.060 g (41%) of the intermediate (6-Methyl-5-(2-methyl-2H-pyrazol-3-yl)-2-oxo-1-(3-trifluoromethyl-phenyl)-1,2-dihydro-pyridine-3-carboxylic acid [3-(3-hydroxy-propyl)-isoxazol-5-ylmethyl]-amide). The hydrolysis was performed in accordance with the hydrolysis in Example 10b, but stirred for 5 hours. Freeze-drying gave 0.033 g (62%) of the title compound as a white solid after purification on HPLC.

¹H NMR (DMSO-d₆): δ 9.83 (1, t, J 6.0 Hz); 8.21 (1H, s); 8.02 (1H, s); 7.92 (1H, d, J 7.4 Hz); 7.87-7.80 (2H, m); 7.53 (1H, d, J 1.8 Hz); 6.33 (1H, d, J 1.8 Hz); 6.20 (1H, s); 4.60 (2H, d, J 6.0 Hz); 4.50 (1H, t, J 5.2 Hz); 3.72 (3H, s); 3.40 (2H, q, J 5.9 Hz); 2.59 (2H, t, J 7.7 Hz); 1.82 (3H, s); 1.70 (2H, p, J 7.4 Hz)

APCI-MS m/z: 516.4 [MH⁺].

### EXAMPLE 13

### 5-(3-Amino-3-oxopropyl)-N-[(3-cyclopropylisoxazol-5-yl)methyl]-6-methyl-2-oxo-1-[3-(trifluoromethyl)phenyl]-1,2-dihydropyridine-3-carboxamide

### a) 5-Iodo-6-methyl-2-oxo-1-(3-trifluoromethyl-phenyl)-1,2-dihydro-pyridine-3-carboxylic acid (3-cyclopropyl-isoxazol-5-Ylmethyl)-amide

Cyclopropane carboxaldehyde (0.7 g, 0.01 mmol) was dissolved in MeOH (25 ml). Hydroxylamine hydrochloride (1.4 g, 0.02 mmol) and NaHCO₃ (1.7 g, 0.02 mmol) was added and the mixture was refluxed overnight, and was then filtered. The filtrate was concentrated in vacuo, and dissolved in CHCl₃ (20 ml). The solution was cooled on an ice-bath under magnetic stirring, NCS(N-chlorosuccinimide, 1.3 g, 0.03 mmol) was added followed by pyridine (4 drops). After completed addition, the ice-bath was removed, and the mixture was stirred for 1 hour. The greenish solution was washed with water and dried. Evaporation of the organic phase gave 1.05 g (75%) of an oil which was used directly in further synthesis. 0.22 g (2 mmol) of this oil was added to a stirred solution of 5-Iodo-6-methyl-2-oxo-1-(3-trifluoromethyl-phenyl)-1,2-dihydro-pyridine-3-carboxylic acid prop-2-ynylamide (Example 1a, 0.15 g, 0.326 mmol) in EtOAc (30 ml). To this mixture, was added water (0.3 ml) and KHCO₃ (0.2 g, 2 mmol). The mixture was heated (45° C.) with stirring overnight. Water was added, the phases were separated, and the organic phase was washed with brine, and dried. Filtration and evaporation gave a crude mixture, which was purified on silica, giving 0.15 g (85%) of the sub-title compound as an amorphous semi-solid material.

APCI-MS m/z: 543.8 [MH⁺].

### b) 3-[5-[(3-Cyclopropyl-isoxazol-5-ylmethyl)-carbamoyl]-2-methyl-6-oxo-1-(3-trifluoromethylphenyl)-1,6-dihydro-pyridin-3-yl]-acrylic acid methyl ester

5-Iodo-6-methyl-2-oxo-1-(3-trifluoromethyl-phenyl)-1,2-dihydro-pyridine-3-carboxylic acid (3-cyclopropyl-isoxazol-5-ylmethyl)-amide (Example 13a, 0.20 g, 0.37 mmol) and Methyl acrylate (0.17 g, 1.9 mmol) was dissolved in CH₃CN (3 ml). To this solution was added Pd(PPh₃)₄ (0.015 g) and TEA (triethylamine, 0.2 ml). The mixture was heated (95° C.) under a nitrogen atmosphere with stirring for 2 hours. The solution was filtered and concentrated in vacuo. Purification on silica gave 0.14 g (75%) of the sub-title compound as a yellowish amorphous solid.

APCI-MS m/z: 502.4 [MH⁺].

### c) 3-[5-[(3-Cyclopropyl-isoxazol-5-ylmethyl)-carbamoyl]-2-methyl-6-oxol-(3-trifluoromethyl-phenyl)-1,6-dihydro-pyridine-3-yl]-propionic acid

3-[5-[(3-Cyclopropyl-isoxazol-5-ylmethyl)-carbamoyl]-2-methyl-6-oxo-1-(3-trifluoromethylphenyl)-1,6-dihydro-pyridin-3-yl]-acrylic acid methyl ester (Example 13b, 0.14 g, 0.28 mmol) was dissolved in EtOAc (5 ml) and EtOH (99.5%, 5 ml), and Pd/C (10%, 0.015 g). The mixture was hydrogenated overnight. The catalyst was removed by filtration. Evaporation gave 0.13 g (92%) of the sub-title compound, This material (0.127 g, 0.25 mmol) was dissolved in THF (5 ml), Water (3 ml) and NaOH (2M, 0.5 ml, 1 mmol). The solution was stirred at room temperature for 1 hour. THF was evaporated, and the residual water solution was diluted with water and acidified with 1M H₂SO₄. Extraction with EtOAc isolated a crude solution of the acid, which was concentrated in vacuo. Purification by preparative HPLC and freeze-drying gave 0.075 g (75%) of the sub-title compound as a white solid.

APCI-MS m/z: 490.4 [MH⁺].

### d) 5-(3-Amino-3-oxopropyl)-N-[(3-cyclopropylisoxazol-5-yl)methyl]-6-methyl-2-oxo-1-[3-trifluoromethyl)phenyl]-1,2-dihydropyridine-3-carboxamide

The compound was prepared according to the method described for Example 10b (final step using 1,4-dioxane and ammonia) starting from 3-[5-[(3-Cyclopropyl-isoxazol-5-ylmethyl)-carbamoyl]-2-methyl-6-oxo1-(3-trifluoromethyl-phenyl)-1,6-dihydro-pyridine-3-yl]-propionic acid (Example 13c) but with ammonia in MeOH instead of aqueous ammonia. Purification on HPLC and freeze-drying gave 0.010 g (80%) of the title compound as a white solid.

¹H NMR (DMSO-d₆): δ 9.90 (1H, t, J 6.0 Hz); 8.32 (1H, s); 7.90 (1H, d, J 7.9 Hz); 7.83 (1H, s); 7.82 (1H, t, J 8.0 Hz); 7.66 (1H, d, J 7.9 Hz); 7.33 (1N, bs), 6.82 (1H, bs); 6.04 (1H, s); 4.55 (2-H, d, J 6.0 Hz); 2.76 (2H, t) J 7.6 Hz); 2.30 (2H, t, J 7.6 Hz); 1.99 (3H, s); 1.99-1.90 (1H, m); 1.00-0.92 (2H, m); 0.74-0.68 (2H, m)

APCI-MS m/z: 489.4 [MH⁺].

### EXAMPLE 14

### 5-(2-Cyanoethyl)-N-[(3-cyclopropylisoxazol-5-yl)methyl]-6-methyl-2-oxo-1-[3-(trifluoromethyl)phenyl]-1,2-dihydropyridine-3-carboxamide

The compound was prepared according to the method described for Example 11 starting from the crude product of 5-(3-Amino-3-oxopropyl)-N-[(3-cyclopropylisoxazol-5-yl)methyl]-6-methyl-2-oxo-1-[3-(trifluoromethyl)phenyl]-1,2-dihydropyridine-3-carboxamide (Example 13d) yielding 0.013 g (56%) of the title compound as a white solid.

¹H NMR (DMSO-d₆): δ 9.87 (1H, t, J 5.8 Hz); 8.41 (1H, s); 7.91 (1H, d, J 7.6 Hz); 7.85 (1H, s); 7.82 (1H, t, J 7.6 Hz); 7.67 (1H, d, J 7.6 Hz); 6.04 (1H, s), 4.55 (2H, d, J 6.0 Hz); 2.91 (2H, t, J 6.8 Hz); 2.74 (2H, t, J 6.8 Hz); 2.98 (3H, s); 1.99-1.90 (1H, m); 1.00-0.91 (2H, m); 0.74-0.67 (2H, m)

APCI-MS m/z: 471.4 [MH⁺].

### EXAMPLE 15

### N-[(3-Cyclopropylisoxazol-5-yl)methyl]-5-[3-(dimethylamino)-3-oxopropyl]-6-methyl-2-oxo-1-[3-(trifluoromethyl)phenyl]-1,2-dihydropyridine-3-carboxamide

The compound was prepared according to the method described for Example 10b (final step using 14-dioxane and ammonia) starting from 3-[5-[(3-Cyclopropyl-isoxazol-5-ylmethyl)-carbamoyl]-2-methyl-6-oxo 1-(3-trifluoromethyl-phenyl)-1,6-dihydro-pyridine-3-yl]-propionic acid (Example 13c) and quenching with dimethylamine, yielding 0.012 g (75%) of the title compound as a white solid.

¹H NMR (DMSO-d₆): δ 9.91 (1H, t, J 6.0 Hz); 8.36 (1H, s); 7.90 (1H, d, J 8.0 Hz); 7.83 (1H, s); 7.81 (1H, t, J 7.8 Hz); 7.66 (1H, d, J 7.9 Hz); 6.04 (1H, s); 4.54 (2H, d, J 6.1 Hz); 2.95 (3H, s); 2.82 (3H, s), 2.76 (2H, t, J 7.5 Hz); 2.56 (2H, t, J 7.5 Hz); 1.99 (3H, s); 1.98-1.90 (1H, m); 0.99-0.92 (2H, m); 0.74-0.67 (2H, m)

### EXAMPLE 16

### 3-{5-({[(3-Cyclopropylisoxazol-5-yl)methyl]amino}carbonyl)-2-methyl-6-oxo-1-[3-(trifluoromethyl)phenyl]-1,6-dihydropyridin-3-yl}propanoic acid

### a) 5-(3-Hydroxy-prop-1-ynyl)-6-methyl-2-oxo-1-(3-trifluoromethyl-phenyl)-1,2-dihydro-pyridine-3-carboxylic acid (3-cyclopropyl-isoxazol-5-ylmethyl)-amide

5-Iodo-6-methyl-2-oxo-1-(3-trifluoromethyl-phenyl)-1,2-dihydro-pyridine-3-carboxylic acid (3-cyclopropyl-isoxazol-5-ylmethyl)-amide (Example 13a, 0.18 g, 0.33 mmol), Propargylalcohol (0.034 g, 0.62 mmol), Pd(PPh₃)₂Cl₂ (0.007 g) and CuI (O 0007 g) was dissolved in Diethylamine (7 ml, 99.5%). The mixture was heated (50° C.) with stirring for 2 hours under nitrogen atmosphere, and was thereafter concentrated in vacuo. The residue was purified by preparative HPLC and freeze-dried giving 0.093 g (60%) of the sub-title compound as a white solid.

APCI-MS m/z: 472.4 [MH⁺].

### b) 3-{5-({[(3-Cyclopropylisoxazol-5-yl)methyl]amino}carbonyl)-2-methyl-6-oxo-1-[3-(trifluoromethyl)phenyl]-1,6-dihydropyridin-3-yl}propanoic acid

5-(3-Hydroxy-prop-1-ynyl)-6-methyl-2-oxo-1-(3-trifluoromethyl-phenyl)-1,2-dihydro-pyridine-3-carboxylic acid (3-cyclopropyl-isoxazol-5-ylmethyl)-amide (Example 16a, 0.093 g, 0.19 mmol) was dissolved in EtOAc (5 ml) and EtOH (99.5%, 5 ml). Pd/C (10%, 0.010 g) was added and the mixture was hydrogenated for 3 hours at room temperature and normal pressure. The filtered solution was concentrated in vacuo. Purification by preparative HPLC, and freeze-drying gave 0.073 g (81%) of the title compound as a white solid.

¹H NMR (DMSO-d₆): δ 9.93 (1H, t, J 6.0 Hz); 8.31 (1H, s); 7.89 (1H, d, J 8.1 Hz); 7.87 (1H, s); 7.81 (1H, t, J 7.8 Hz); 7.68 (1H, d, J 7.7 Hz); 6.04 (1H, s); 4.55 (2H, d, J 6.0 Hz); to 4.52 (1H, t, J 5.1 Hz); 3.45 (2H, q, J 5.8 Hz); 2.58 (2H, t, J 7.6 Hz); 1.98 (3H, s); 1.98-1.90 (1H, m); 1.65 (2H, p, J 6.9 Hz); 0.99-0.92 (2H, m); 0.73-0.68 (2H, m)

APCI-MS m/z: 476.4[MH⁺]. Retention time 2.17 minutes

### EXAMPLE 17

### N-[(3-Cyclopropylisoxazol-5-yl)methyl]-6-methyl-5-[3-(methylsulfonyl)propyl]-2-oxo-1-[3-(trifluoromethyl)phenyl]-1,2-dihydropyridine-3-carboxamide

### a) Methanesulfonic acid 3-[5-[(3-Cyclopropyl-isoxazol-5-ylmethyl)-carbamoyl]-2-methyl-6-oxo-1-(3-trifluoromethyl-phenyl-1,6-dihydro-pyridin-3-yl]-propyl ester

3-{5-({[(3-Cyclopropylisoxazol-5-yl)methyl]amino}carbonyl)-2-methyl-6-oxo-1-[3-(trifluoromethyl)phenyl]-1,6-dihydropyridin-3-yl}propanoic acid (Example 16, 0.070 g, 0.147 mmol) was dissolved in 1,4-Dioxane (dry, 5 ml). Hunigs base (DIPEA, 0.050 g, 0.38 mmol) and Methanesulfonyl chloride (0.050 g, 0.4 mmol) was added and the mixture was allowed to stand at room temperature for 1 hour, and then concentrated in vacuo. The residue was purified by preparative HPLC and freeze-dried giving 0.038 g (47%) of the sub-title compound as a white solid.

APCI-MS m/z: 554.4 [MH⁺].

### b) 6-Methyl-5-(3-methylsulfanyl-propyl)-2-oxo-1-(3-trifluoromethyl-phenyl)-1,2-dihydro-pyridine-3-carboxylic acid (3-cyclopropyl-isoxazol-5-ylmethyl)-amide

Methanesulfonic acid 3-[5-[(3-Cyclopropyl-isoxazol-5-ylmethyl)-carbamoyl]-2-methyl-6-oxo-1-(3-trifluoromethyl-phenyl)-1,6-dihydro-pyridin-3-yl]-propyl ester (Example 17a, 0.037 g, 0.067 mmol) was dissolved in DMF (1.5 ml). Sodium Methanethiolate (NaSMe, 0.012 g, 0.156 mmol) was added and the mixture was stirred at room temperature for 1 hour. Purification on preparative HPLC, and evaporation yielded 0.026 g (77%) of the sub-title compound as a yellowish oil.

APCI-MS m/z: 506.4 [MH⁺].

### c) N-[(3-Cyclopropylisoxazol-5-yl)methyl]-6-methyl-5-[3-(methylsulfonyl)propyl]-2-oxo-1-[3-(trifluoromethyl)phenyl]-1,2-dihydropyridine-3-carboxamide

6-Methyl-5-(3-methylsulfanyl-propyl)-2-oxo-1-(3-trifluoromethyl-phenyl)-1,2-dihydro-pyridine-3-carboxylic acid (3-cyclopropyl-isoxazol-5-ylmethyl)-amide (Example 17b, 0.022 g, 0.044 mmol) was dissolved in CH₂Cl₂ (5 ml). m-CPBA (m-Chloroperoxybenzoic acid, 0.025 g, approximately 0.10 mmol) was added and the solution was stirred at room temperature for 1 hour. The mixture was concentrated in vacuo, and was purified by preparative HPL. Freeze-drying gave 0.021 g (89%) of the title compound as a white solid.

¹H NMR (DMSO-d₆): δ 9.92 (1H, t, J 6.0 Hz); 8.34 (1H, s); 7.90 (1H, d, J 7.8 Hz); 7.87 (1H, s); 7.81 (1H, t, J 7.8 Hz); 7.69 (1H, d, J 7.8 Hz); 6.04 (1H, s); 4.55 (2H, d, J 6.0 Hz); 3.23-3.25 (2H, m); 2.97 (3H, s); 2.73-2.66 (2H, m); 1.99 (3H, s); 1.97-1.86 (3H, m); 0.99-0.92 (2H, m); 0.74-0.68 (2H, m).

APCI-MS m/z: 538.4 [MH⁺].

### EXAMPLE 18

### N-[(3-Cyclopropylisoxazol-5-yl)methyl]-6-methyl-5-{3-[(methylsulfonyl)amino]propyl}-2-oxo-1-[3-(trifluoromethyl)phenyl]-1,2-dihydropyridine-3-carboxamide

### a) {3-[5[(3-Cyclopropyl-isoxazol-5-ylmethyl)-carbamoyl]-2-methyl-6-oxo-1-(3-trifluoromethyl-phenyl)-1,6-dihydro-pyridin-3-yl]-propyl}-carbamic acid tert-butyl ester

The compound was prepared in two steps; The first step was performed according to the method described for Example 16a starting from 5-iodo-6-methyl-2-oxo-1-(3-trifluoromethyl-phenyl)-1,2-dihydro-pyridine-3-carboxylic acid (3-cyclopropyl-isoxazol-5-ylmethyl)-amide (Example 13a) and propargylamine-N-tert-butyl carbamate. Purification on silica afforded 0.09 g of the intermediate as an amorphous solid. The second step, hydrogenation for 12 hrs, was performed according to the method described for Example 16b but without purification. Instead the filtrate was concentrated in vacuo, giving 0.09 g (77%) of the sub-title compound as a yellowish amorphous solid.

APCI-MS m/z: 575.1 [MH⁺].

### b) 5-(3-Amino-propyl)-6-methyl-2-oxo-1-(3-trifluoromethyl-phenyl)-1,2-dihydro-pyridine-3-carboxylic acid (3-cyclopropyl-isoxazol-5-ylmethyl)-amide

{3-[5[(3-Cyclopropyl-isoxazol-5-ylmethyl)-carbamoyl]-2-methyl-6-oxo-1-(3-trifluoromethyl-phenyl)-1,6-dihydro-pyridin-3-yl]-propyl}-carbamic acid tert-butyl ester (Example 18a, 0.09 g, 0.156 mmol) was dissolved in CH₂Cl₂ (5 ml) and TFA (1 ml). The mixture was allowed to stand for 1 hour followed by concentration in vacuo giving the sub-title compound as a brownish oil, which was used in synthesis without purification.

APCI-MS m/z: 475.5 [MH⁺].

### c) N-[(3-Cyclopropylisoxazol-5-yl)methyl]-6-methyl-5-{3-[(methylsulfonyl)amino]propyl}-2-oxo-1-[3-(trifluoromethyl)phenyl]-1,2-dihydropyridine-3-carboxamide

5-(3-Amino-propyl)-6-methyl-2-oxo-1-(3-trifluoromethyl-phenyl)-1,2-dihydro-pyridine-3-carboxylic acid (3-cyclopropyl-isoxazol-5-ylmethyl)-amide (Example 18b, 0.039 mmol) was dissolved in CH₂Cl₂ (2 ml), Hunigs base (DIPEA, 5 drops) and Methanesulfonyl chloride (3 drops). The mixture was allowed to stand for 20 minutes and concentrated in vacuo. Purification by preparative HPLC and freeze-drying gave 0.009 g (42%) of the title compound as a white solid.

¹H NMR (DMSO-d₆): δ 9.92 (1H, t, J 6.0 Hz); 8.34 (1H, s); 7.89 (1H, d, J 7.9 Hz); 7.86 (1H, s); 7.81 (1H, t, J 7.7 Hz); 7.68 (1H, d, J 7.8 Hz); 7.06 (1H, t, J 5.7 Hz); 6.04 (1H, s); 4.55 (2H, d, J 6.0 Hz); 3.01 (2H, q, J 6.2 Hz); 2.90 (3H, s); 2.63-2.56 (2H, m); 1.98 (3H, s); 1.98-1.90 (1H, ma); 1.70 (2H, p, J 7.5 Hz); 0.99-0.90 (2H, m); 0.74-0.67 (2H, m)

APCI-MS m/z: 553.4 [MH⁺].

### INTERMEDIATE EXAMPLE 6

### 5-Iodo-6-methyl-N-[4-(methylsulfonyl)benzyl]-2-oxo-1-[3-(trifluoromethyl)phenyl]-1,2-dihydropyridine-3-carboxamide

### a) 6-Methyl-N-[4-(methylsulfonyl)benzyl]-2-oxo-1-[3-(trifluoromethyl)phenyl]-1,2-dihydropyridine-3-carboxamide

A mixture of 6-methyl-2-oxo-1-[3-(trifluoromethyl)phenyl]-1,2-dihydropyridine-3-carboxylic acid (Intermediate Example 1b 7.43 g, 25 mmol), HATU (10.5 g, 27.5 mmol), HOAT (3.75 g, 27.5 mmol) and DIEA (14.2 ml, 82.5 mmol) in NMP (65 ml) was reacted for 1 hour, then 4-methylsulphonylbenzyl amine hydrochloride (5.8 g, 26 mmol) was added. After 1 hour, the reaction mixture was slowly poured into stirred ice water (1 L). A powder was formed, and the water mixture was acidified to pH 3 with citric acid (0.5 M), and stirring was continued for 1 hour. The precipitate was filtered off, washed with water and dried in vacuum overnight. Recrystallisation from EtOAc gave 8.1 g (70%) of the sub-title compound.

¹H NMR (CDCl₃): δ 10.00 (1H, brt); 8.60 (1H, d); 7.88 (2H, d); 7.83 (1H, d); 7.76 (1H, t); 7.53 (3H, m); 7.46 (1H, d); 6.49 (1H, d); 4.68 (2H, m); 3.03 (3H, s); 2.10 (3H, s). APCI-MS m/z: 465.1 [MH⁺].

### b) 5-Iodo-6-methyl-N-[4-(methylsulfonyl)benzyl]-2-oxo-1-[3-(trifluoromethyl)phenyl]-1,2-dihydropyridine-3-carboxamide

To a solution of 6-methyl-N-[4-(methylsulfonyl)benzyl]-2-oxo 1-[3-(trifluoromethyl)phenyl]-1,2-dihydropyridine-3-carboxamide (Intermediate Example 6a, 200 mg, 0.43 mmol) in MeCN (1.5 ml) at room temperature and under argon was added trifluoromethanesulfonic acid (1 ml) followed by N-iodosuccinimide (97 mg, 0.43 mmol). After 45 minutes, the reaction mixture was diluted with DCM, washed with aqueous NaHCO₃, with aqueous NaS₂O₄ and water, dried (Na₂SO₄), and evaporated to give the title compound (200 mg).

¹H NMR (CDCl₃): δ 9.85 (1H, brt); 8.90 (1H, d); 7.88 (2H, d); 7.76 (2H, m); 7.50 (2H, d); 7.48 (1H, s); 7.40 (1H, d); 4.65 (2H, m); 3.03 (3H, s); 2.32 (3H, s).

APCI-MS m/z: 591.0 [MH⁺].

### INTERMEDIATE EXAMPLE 7

### N-[4-(Cyclopropylsulfonyl)benzyl]-5-iodo-6-methyl-2-oxo-1-[3-(trifluoromethyl)phenyl]-1,2-dihydropyridine-3-carboxamide

The title compound was prepared using a procedure analogous to that described for Intermediate Example 6.

¹H NMR (CDCl₃): δ 9.86 (1H, t, J 5.8 Hz); 8.90 (1H, s); 7.83-7.80 (3H, m); 7.75 (1H, t, J 7.8 Hz); 7.49-7.47 (3H, m); 7.40 (1H, d, J 7.8 Hz); 4.66 (2H, t, J 5.7 Hz); 2.42 (1H, m); 2.31 (3H, s); 1.32 (2H, m); 1.01 (2H, m).

APCI-MS m/z: 617 [MH⁺].

### EXAMPLE 19

### 6-Methyl-5-{3-[(methylsulfonyl)amino]propyl}-N-[4-(methylsulfonyl)benzyl]-2-oxo-1-[3-(trifluoromethyl)phenyl]-1,2-dihydropyridine-3-carboxamide

The title compound was prepared according to the procedures described for Example 18 starting from 5-Iodo-6-methyl-N-[4-(methylsulfonyl)benzyl]-2-oxo-1-[3-(trifluoromethyl)phenyl]-1,2-dihydropyridine-3-carboxamide (Intermediate Example 6).

¹H NMR (DMSO-d₆): δ 10.00 (1H, t, J 6.0 Hz); 8.35 (1H, s); 7.91-7.84 (4H, m); 7.80 (1H, t, J 7.8 Hz); 7.68 (1H, d, J 7.8 Hz); 7.53 (2H, d, J 8.0 Hz); 7.06 (1H, t, J 5.7 Hz); 4.58 (2H, d, J 6.0 Hz); 3.17 (3H, 5s); 3.01 (2H, q, J 6.1 Hz); 2.90 (3H, s); 2.63-2.56 (2H, m); 1.98 (3H, s); 1.70 (2H, p, J 7.5 Hz)

APCI-MS m/z: 600.4 [MH⁺].

### EXAMPLE 20

### 5-(3-Hydroxyprop-1-yn-1-yl)-6-methyl-N-[4-(methylsulfonyl)benzyl]-2-oxo-1-[3-(trifluoromethyl)phenyl]-1,2-dihydropyridine-3-carboxamide

The compound was prepared according to the procedure described for Example 16a, starting from 5-Iodo-6-methyl-N-[4-methylsulfonyl)benzyl]-2-oxo-1-[3-(trifluoromethyl)phenyl]-1,2-dihydropyridine-3-carboxamide (Intermediate Example 6, 0.10 g, 0.169 mmol). Following this method, 0.064 g (73%) was obtained of the title compound as a white solid.

¹H NMR (DMSO-d₆): δ 9.78 (1H, t, J 6.1 Hz); 8.31 (1H, s); 7.95 (1H, s); 7.91 (1H, d, J 8.1 Hz); 7.86 (2H, d, J 8.2 Hz); 7.83 (1H, t, J 7.8 Hz); 7.76 (1H, d, J 7.8 Hz); 7.53 (2H, d, J 58.2 Hz); 5.34 (1H, t, J 5.9 Hz); 4.58 (2H, d, J 6.1 Hz); 4.33 (2H, d, J 6.0 Hz); 3.17 (3H, s); 2.16 (3H, s)

APCI-MS m/z: 518.8 [MH⁺].

### EXAMPLE 21

### 5-(3-Amino-3-oxopropyl)N-[4-(cyclopropylsulfonyl)benzyl]-6-methyl-2-oxo-1-[3-(trifluoromethyl)phenyl]-1,2-dihydropyridine-3-carboxamide

The compound was prepared according to procedures described in Example 13a-13d starting from N-[4-(cyclopropylsulfonyl)benzyl]-5-iodo-6-methyl-2-oxo-1-[3-(trifluoromethyl)phenyl]-1,2-dihydropyridine-3-carboxamide (intermediate Example 7). This gave 0.015 g (75%) of the title compound as a white solid.

¹H NMR (DMSO-d₆): δ 9.99 (1H, t, J 6.1 Hz); 8.33 (1H, s); 7.89 (1H, d, J 7.9 Hz); 7.85-7.78 (4H, m); 7.67 (1H, d, J 7.9 Hz); 7.53 (2H, d, J 8.2 Hz); 7.33 (1H, bs); 6.82 (1H, bs); 4.58 (2H, d, J 6.1 Hz); 2.84-2.74 (1H, m); 2.76 (2H, t, J 7.7 Hz); 2.31 (2H, t, J 7.6 Hz); 1.99 (3H, s); 1.11-1.06 (2H, m); 1.05-0.97 (2H, m)

APCI-MS m/z: 562.4 [MH⁺].

### INTERMEDIATE EXAMPLE 8

### 5-Hydroxy-N-[4-(isopropylsulfonyl)benzyl]-6-methyl-2-oxo-1-[3-(trifluoromethyl)phenyl]-1,2-dihydropyridine-3-carboxamide

### a) N-[4-(Isopropylsulfonyl)benzyl]-6-methyl-2-oxo-1-[3-(trifluoromethyl)phenyl]-1,2-dihydropyridine-3-carboxamide

To a mixture of 6-methyl-2-oxo-1-[3-(trifluoromethyl)phenyl]-1,2-dihydropyridine-3-carboxylic acid (Intermediate Example 1b, 16.27 g, 54.5 mmol) in DCM was added thionyl chloride (12 ml, 165 mmol) under argon. After 50 minutes stirring at ambient temperature, the solvent was removed by evaporation. The last traces of thionyl chloride were removed by azeotropic evaporation with toluene. To an ice cooled solution of the residue in DCM, was added dropwise a mixture of 1-[4-(isopropylsulfonyl)phenyl]methanamine (11.8 g, 55.4 mmol) and triethylamine (30 ml, 215 mmol) in DCM under vigorous stirring. After the addition, the dark suspension was allowed to warm up to room temperature. After 30 minutes stirring at ambient temperature the reaction mixture was concentrated in vacuo and the residue was partitioned between ethyl acetate and water. The organic layer was washed with water, dried over sodium sulfate, filtered and concentrated in vacuo, giving a dark oil which crystallized on standing. The solid was triturated with ethyl acetate, filtered, washed with ethyl acetate, ether, heptane, and dried under vacuo to give the title compound as a light yellow powder (15.3 g). The filtrates were collected, concentrated and further purified by flash chromatography on silica, eluting with a gradient of tert-butyl methyl ether to 5% methanol in tert-butyl methyl ether to provide an additional 8.78 g of the crude product. The solids were combined to give (24.1 g, 89%) of the sub-title compound.

¹H NMR (CDCl₃): δ 9.96 (1H, t, J 5.5 Hz); 8.57 (1H, d, J 7.4 Hz); 7.78 (3H, t, J 4.1 Hz); 7.72 (1H, t, J 7.9 Hz); 7.52-7.45 (3H, m); 7.43 (1H, d, J 7.7 Hz); 6.46 (1H, d, J 7.6 Hz); 4.67 (2H, ddd, J 22.0 15.7 6.2 Hz); 3.13 (1H, septet, J 9.8 Hz); 2.07 (3H, s); 1.26 (6H, d, J 6.9 Hz).

APCI-MS m/z: 493.2 [MH⁺].

### b) 5-Iodo-N-[4-(isopropylsulfonyl)benzyl]-6-methyl-2-oxo-1-[3-(trifluoromethyl)phenyl]-1,2-dihydropyridine-3-carboxamide

To a stirred solution of N-[4-(isopropylsulfonyl)benzyl]-6-methyl-2-oxo-1-[3-(trifluoromethyl)phenyl]-1,2-dihydropyridine-3-carboxamide (Intermediate Example 8a, 23.98 g, 48.73 mmol) and TFA (90 ml) in DCM (90 ml) was added N-iodosuccinimide (11.03 g, 49.14 mmol). After 2 hours the reaction was complete and the solvent was removed by evaporation. To the residue was added ethyl acetate (100 ml) and saturated aqueous sodium hydrogencarbonate solution (60 ml) under stirring. The yellow solid was collected by suction filtration, washed with water, air dried for 30 minutes, washed again with diethyl ether, heptane and vacuum dried to give the sub-title compound as a light yellow powder (29.67 g, 98%).

¹H NMR (CDCl₃): δ 9.83 (1H, t, J 6.0 Hz); 8.90 (1H, s); 7.83-7.76 (3H, m); 7.73 (1H, t, J 7.8 Hz); 7.47 (3H, d, J 8.0 Hz); 7.39 (1H, d, J 7.7 Hz); 4.66 (2H, ddd, J 22.3, 15.8 and 6.3 Hz); 3.13 (1H, septet, 19.0 Hz); 2.29 (3H, s); 1.26 (6H, d, J 6.9 Hz).

APCI-MS m/z: 619.1 [MH⁺].

### c) 5-Acetyl-1-N-[4-(isopropylsulfonyl)benzyl]-6-methyl-2-oxo-1-[3-(trifluoromethyl phenyl]-1,2-dihydropyridine-3-carboxamide

A mixture of 5-iodo-N-[4-(isopropylsulfonyl)benzyl]-6-methyl-2-oxo-1-[3-(trifluoromethyl)phenyl]-1,2-dihydropyridine-3-carboxamide (Intermediate Example 8b, 3.55 g, 5.7 mmol), bis[1,2-bis(diphenylphosphino)ethane]-palladium (0) (24.5 mg, 0.03 mmol), n-butyl vinyl ether (1.16 g, 11.6 mmol), triethylamine (4 ml, 28.7 mmol) in DMF (14 ml) was stirred at 100° C. under argon for 19 hours. The reaction mixture was cooled and concentrated in vacuo. The residue was dissolved in methanol (20 ml) and 2M hydrochloric acid (2 ml) was added. After 1 hour stirring at room temperature the mixture was partitioned between ethyl acetate/water and basified with saturated aqueous sodium bicarbonate solution. The water layer was extracted with ethyl acetate (2×50 ml) and DCM (1×30 ml). The combined organic layers were washed with water, brine, dried over sodium sulfate, filtered and concentrated in vacuo. The residue was purified by flash chromatography on silica eluting with tert-butyl methyl ether/methanol (10:0.2) to give the sub-title compound as a light yellow solid (2.5 g, 82%).

¹H NMR (CDCl₃): δ 9.71 (1H, t, J 5.7 Hz); 9.05 (1H, s); 7.85-7.78 (3H, m); 7.75 (1H, t, J 7.9 Hz); 7.51-7.44 (3H, m); 7.39 (1H, d, J 8.2 Hz); 4.68 (2H, ddd, J 22.4, 15.8 and 6.2 Hz); 3.14 (1H, septet, J 7.7 Hz); 2.63 (3H, s); 2.40 (3H, s); 1.26 (6H, d, J 6.9 Hz).

APCI-MS m/z: 535.2 [MH⁺].

### d) 5-Hydroxy-N-[4-(isopropylsulfonyl)benzyl]-6-methyl-2-oxo-1-[3-(trifluoromethyl)phenyl]-1,2-dihydropyridine-3-carboxamide

To ice-cooled 35% hydrogen peroxide (11.11 g, 114.4 mmol) was added concentrated sulphuric acid (8.92 g, 91.0 mmol) and 5-acetyl-N-[4-(isopropylsulfonyl)benzyl]6-methyl-2-oxo-1-[3-(trifluoromethyl)phenyl]-1,2-dihydropyridine-3-carboxamide (Intermediate Example 8c, 2.2 g, 4.1 mmol) in DCM (3 ml). The mixture was stirred vigorously and heated at 45° C. for 1.5 hours. The reaction mixture was cooled to room temperature and then added dropwise to an ice cooled mixture of ethyl acetate (100 ml) and saturated aqueous sodium carbonate solution under stirring. The organic layer was collected and the water layer was extracted with ethyl acetate (2×60 ml). The combined organic layers were washed with water and brine, dried over sodium sulfate, filtered and concentrated in vacuo. The residue was purified by flash chromatography on silica eluting with tert-butyl methyl ether/methanol (10:0.2) to give the title compound as a yellow solid (1.1 g, 52%).

¹H NMR (CDCl₃): δ 10.66 (1H, t, J 6.0 Hz); 8.97 (1H, s); 8.16 (1H, s); 7.81 (3H, m); 7.74 (1H, t, J 7.8 Hz); 7.51 (3H, t, J 4.1 Hz); 7.43 (1H, d, J 7.8 Hz); 4.68 (2H, td, J 9.5 4.5 Hz); 3.16 (1H, quintet, J 6.9 Hz); 2.04 (3H, s); 1.28 (6H, d, J 6.9 Hz).

APCI-MS m/z: 509.1 [MH⁺].

### EXAMPLE 22

### N-[4-(Isopropylsulfonyl)benzyl]-6-methyl-5-(2-morpholin-4-ylethoxy)-2-oxo-1-[3-(trifluoromethyl)phenyl]-1,2-dihydropyridine-3-carboxamide

### a) 5-(2-Bromoethoxy)-N-[4-(isopropylsulfonyl)benzyl]-6-methyl-2-oxo-1-[3-(trifluoromethyl)phenyl]-1,2-dihydropyridine-3-carboxamide

To a mixture of 5-hydroxy-N-[4-(isopropylsulfonyl)benzyl]-6-methyl-2-oxo-1-[3-(trifluoromethyl)phenyl]-1,2-dihydropyridine-3-carboxamide (Intermediate Example 8, 500 mg, 0.98 mmol) and cesium carbonate (1.28 g, 3.94 mmol) in DMF, 1.3 dibromopropane (795 mg, 3.94 mmol) was added and the mixture was heated to 70° C. for 0.5 hour. The compound was then purified on preparative HPLC. Freeze drying of the mixture afforded the title compound (100 mg, 16%).

APCI-MS m/z: 629.2 [MH⁺].

### b) N-[4-(Isopropylsulfonyl)benzyl]-6 methyl-5-(2-morpholin-4-ylethoxy)-2-oxo-1-[3-(trifluoromethyl)phenyl]-1,2-dihydropyridine-3-carboxamide

A solution of 5-(2-bromoethoxy)-N-[4-(isopropylsulfonyl)benzyl]-6-methyl-2-oxo-1-[3-(trifluoromethyl)phenyl]-1,2-dihydropyridine-3-carboxamide (Example 22a, 23 mg, 0.036 mmol) and morpholine (32 mg, 0.36 mmol) was heated in a microwave for 5 minutes at 50° C. The mixture was purified by preparative HPLC to give the title compound as a white solid (3 mg, 13%).

¹H NMR (300 MHz, CD₂Cl₂) δ 10.10 (H, t, J 5.9 Hz); 8.40 (1H, s); 7.90 (2H, d); 7.82 (3H, dd, J 11.6, 8.3 Hz); 7.72 (1H, d, J 8.2 Hz); 7.54 (2H, d, J 8.2 Hz); 4.61 (2H, d, J 6.0 Hz); 4.03 (2H, t); 3.62 (3H, s,); 3.39 (3H, t, J 6.7 Hz); 1.96 (3H, s); 1.13 (6H, d, J 6.8 Hz)

APCI-MS m/z: 636.3 [MH⁺].

### INTERMEDIATE EXAMPLE 9

### 5-(1-Hydroxyethyl)-6-methyl-N-[4-(methylsulfonyl)benzyl]-2-oxo-1-[3-(trifluoromethyl)phenyl]-1,2-dihydropyridine-3-carboxamide

### a) 5-(1-Butoxyvinyl)-6-methyl-N-[4-(methylsulfonyl)benzyl]-2-oxo-1-[3-(trifluoromethyl)phenyl]-1,2-dihydropyridine-3-carboxamide

In a Schlenk vessel equipped with a magnetic stirring bar were placed 5-iodo-6-methyl-N-[4-(methylsulfonyl)benzyl]-2-oxo-1-[3-(trifluoromethyl)phenyl]-1,2-dihydropyridine-3-carboxamide (Intermediate Example 6, 101.5 mg, 0.17 mmol), bis[1,2-bis(diphenylphosphino)ethane]-palladium (0) (16.5 mg, 18.3 μmol), n-butyl vinyl ether (60 μl, 0.46 mmol), triethylamine (0.5 ml, 3.6 mmol) and DMF (6 ml). The vessel was purged with argon, sealed and heated at 100° C. overnight. The reaction mixture was cooled and partitioned between ethyl acetate and water. The organic layer was dried over sodium sulphate, filtered and concentrated in vacuo. The residue was purified by preparative HPLC to give the sub-title compound as a white solid (27.3 mg, 28%).

¹H NMR (CDCl₃): δ 9.96 (1H, t, J 5.8 Hz); 8.64 (1H, s); 7.89 (2H, d, J 8.3 Hz); 7.82 (1H, d, J 8.0 Hz); 7.75 (1H, t, J 7.9 Hz); 7.56-7.50 (3H, m); 7.46 (1H, d, J 7.8 Hz); 4.69 (2H, ddd, J 22.1, 15.7, 6.2 Hz); 4.43 (1H, d, J 2.6 Hz); 4.26 (1H, d, J 2.6 Hz); 3.83 (2H, t, J 6.5 Hz); 3.03 (3H, s); 2.11 (3H, s); 1.74 (2H, quintet, J 9.2 Hz); 1.46 (2H, sextet, J 9.1 Hz); 0.98 (3H, t, 37.4 Hz).

APCI-MS m/z: 563 [MH⁺].

### b) 5-Acetyl-6-methyl-N-[4-(methylsulfonyl)benzyl]-2-oxo-1-[3-(trifluoromethyl)phenyl]-1,2-dihydropyridine-3-carboxamide

To a solution of 5-(1-butoxyvinyl)-6-methyl-N-[4-(methylsulfonyl)benzyl]-2-oxo-1-[3-(trifluoromethyl)phenyl]-1,2-dihydropyridine-3-carboxamide (Intermediate Example 9a, 38 mg, 67.5 μmol) in DMF (0.5 ml) was added aqueous hydrochloric acid (2.0M, 50 μl). After 20 min. the solution was neutralized with aqueous sodium hydrogen carbonate. The reaction mixture was purified by preparative HPLC to give the sub-title compound as a white solid (17.6 mg, 51%).

¹H NMR (CDCl₃): δ 9.75 (1H, t, J 5.7 Hz); 9.08 (1H, s); 7.90 (2H, d, J 8.3 Hz); 7.85 (1H, d, J 7.9 Hz); 7.78 (1H, t, J 7.9 Hz); 7.54 (2H, d, J 8.3 Hz); 7.50 (1H, s); 7.42 (1H, d, J 8.0 Hz); 4.70 (2H, t, J 6.0 Hz); 3.03 (3H, s); 2.66 (3H, s); 2.43 (3H, s).

APCI-MS m/z: 507 [MH⁺].

### c) 5-(1-Hydroxyethyl)-6-methyl-N-[4-(methylsulfonyl)benzyl]-2-oxo-1-[3-(trifluoromethyl)phenyl]-1,2-dihydropyridine-3-carboxamide

A mixture of 5-acetyl-6-methyl-N-[4-(methylsulfonyl)benzyl]-2-oxo-1-[3-(trifluoromethyl)phenyl]-1,2-dihydropyridine-3-carboxamide (Intermediate Example 9b, 180 mg, 0.35 mmol) and aluminum tri-sec-butoxide (0.2 mg, 0.79 mmol) in anhydrous isopropanol (30 ml) was stirred at 85° C. under a nitrogen atmosphere for 48 hours. The reaction mixture was cooled to room temperature, water (0.2 ml) was added and the mixture was then concentrated in vacuo. The residue was purified by preparative HPLC to give the title compound as a white solid (134 mg, 74%).

¹H NMR (CDCl₃): δ 10.01 (1H, t, J 5.7 Hz); 8.84 (1H, d, J 1.9 Hz); 7.87 (2H, d, J 8.3 Hz); 7.81 (1H, d, J 7.8 Hz); 7.74 (1H, t, J 8.0 Hz); 7.52 (2H, d, J 8.3 Hz); 7.50 (1H, s); 7.42 (1H, d, J 7.9 Hz); 5.03 (1H, dd, J 10.9 1.6 Hz); 4.67 (2H, q, J 6.3 Hz); 3.02 (3H, s); 0.12 (3H, s); 1.91 (1H, t, J 3.9 Hz); 1.58 (3H, dd, J 6.4 2.6 Hz).

APCI-MS m/z: 509.2 [MH⁺].

### INTERMEDIATE EXAMPLE 10

### 5-(Chloromethyl)-6-methyl-N-[4-(isopropylsulfonyl)benzyl]-2-oxo-1-[3-(trifluoromethyl)phenyl]-1,2-dihydropyridine-3-carboxamide

### a) 5-(Hydroxymethyl)-6-methyl-N-[4-(isopropylsulfonyl)benzyl]-2-oxo-1-[3-(trifluoromethyl)phenyl]-1,2-dihydropyridine-3-carboxamide

The title compound was prepared using a procedure analogous to that described for Intermediate Example 9c.

### b) 5-(Chloromethyl)-6-methyl-N-[4-(isopropylsulfonyl)benzyl]-2-oxo-1-[3-(trifluoromethyl)phenyl]-1,2-dihydropyridine-3-carboxamide

5-(Hydroxymethyl)-6-methyl-N-[4-(isopropylsulfonyl)benzyl]-2-oxo-1-[3-(trifluoromethyl)phenyl]-1,2-dihydropyridine-3-carboxamide (Intermediate Example 10a) was treated with thionyl chloride to give the title compound.

### EXAMPLE 23

### N-[4-(Cyclopropylsulfonyl)benzyl]-6-methyl-5-[(methylsulfonyl)methyl]-2-oxo-1-[3-(trifluoromethyl)phenyl]-1,2-dihydropyridine-3-carboxamide

A mixture of 5-(chloromethyl)-6-methyl-N-[4-(methylsulfonyl)benzyl]-2-oxo-1-[3-(trifluoromethyl)phenyl]-1,2-dihydropyridine-3-carboxamide (Intermediate Example 10, 103 mg, 0.192 mmol) and sodium methanesulfinate (29.5 mg, 0.288 mmol) in DMSO (2 ml) was heated at 50° C. under argon overnight. The mixture was purified by preparative HPLC to give the title compound as a white solid (18 mg, 16%).

¹H NMR (400 MHz, dmso): δ 9.89 (1H, t, J 6.0 Hz); 8.47 (1H, s) 7.91 (3H, t, J 7.7 Hz); 7.82 (3H, t, J 8.2 Hz); 7.72 (1H, d, J 7.8 Hz); 7.53 (2H, d, J 8.3 Hz); 4.60 (4H, m); 3.57 (3H, s,); 3.04 (3H, s,); 2.80 (1H, m); 2.08 (3H, s); 1.09 (2H, m); 1.01 (2H, m);

APCI-MS m/z: 583.3 [MH⁺].

### EXAMPLE 24

### 5-(1-Cyanoethyl)-6-methyl-N-[4-(methylsulfonyl)benzyl]-2-oxo-1-[3-(trifluoromethyl)phenyl]-1,2-dihydropyridine-3-carboxamide

To a solution of 5-(1-hydroxyethyl)-6-methyl-N-[4-(methylsulfonyl)benzyl]-2-oxo-1-[3-(trifluoromethyl)phenyl]-1,2-dihydropyridine-3-carboxamide (Intermediate Example 9, 71 mg, 0.03 mmol) in dichloromethane (2.5 ml) was added thionyl chloride (0.5 ml, 6.9 mmol) under argon atmosphere. After 2 hours stirring at ambient temperature, the solvent was removed in vacuum. The last traces of thionyl chloride were removed by azeotropic evaporation with toluene. The residue was dissolved in anhydrous N,N-dimethylformamide (2 ml) and potassium cyanate (113 mg, 1.8 mmol) was added. After 20 minutes stirring at 55° C. the reaction mixture was diluted with water and further purified by preparative HPLC giving the title compound as a white solid (59.8 mg, 82%).

¹H NMR (CDCl₃): δ 9.88 (1H, t, J 5.7 Hz); 8.71 (1H, d, J 6.7 Hz); 7.88 (3H, d, J 8.3 Hz); 7.84 (3H, d, J 7.9 Hz); 7.77 (1H, mult); 7.52 (3H, d, J 8.3 Hz); 7.49 (3H, s); 7.43 (211, t, J 9.3 Hz); 4.68 (2H, mult); 3.95 (1H, q, J 7.3 Hz); 3.02 (3H, s); 2.16 (3H, d, 33.0 Hz); 1.73 (3H, dd, J 7.3, 2.9 Hz).

APCI-MS m/z: 518.1 [MH⁺].

### EXAMPLE 25

### Ethyl 3-{5-({[4-(cyclopropylsulfonyl)benzyl]amino}carbonyl)-2-methyl-6-oxo-1-[3-(trifluoromethyl)phenyl]-1,6-dihydropyridin-3-yl}propanoate

### a) Ethyl (2E)-3-{5-({[4-(cyclopropylsulfonyl)benzyl]amino}carbonyl)-2-methyl-6-oxo-1-[3-(trifluoromethyl)phenyl]-1,6-dihydropyridin-3-yl}acrylate

The title compound was prepared according to the procedure described in Example 13b starting from N-[4-(Cyclopropylsulfonyl)benzyl]-5-iodo-6-methyl-2-oxo-1-[3-(trifluoromethyl)phenyl]-1,2-dihydropyridine-3-carboxamide (Intermediate Example 7).

APCI-MS m/z: 589.6 [MH⁺].

### b) Ethyl 3-{5-({[4-(cyclopropylsulfonyl)benzyl]amino}carbonyl)-2-methyl-6-oxo-1-[3-(trifluoromethyl)phenyl]-1,6-dihydropyridin-3-yl}propanoate

A mixture of ethyl (2E)-3-{5-({[4-(cyclopropylsulfonyl)benzyl]amino}carbonyl)-2-methyl-6-oxo-1-[3-(trifluoromethyl)phenyl]-1,6-dihydropyridin-3-yl}acrylate (Example 25a, 157.6 mg, 0.27 mmol), 5% palladium on carbon (15.6 mg) in ethanol (10 ml) and ethyl acetate (10 ml) was stirred vigorously under a hydrogen atmosphere for 6 hours. The mixture was filtered through celite, the filtrate was evaporated to dryness and the residue was purified by preparative HPLC to give the title compound as a white solid (132.3 mg, 82%).

¹H NMR (CDCl₃): δ 10.01 (1H, t, J 5.8 Hz); 8.54 (1H, s); 7.82 (2H, d, J 8.4 Hz); 7.79 (1H, s); 7.74 (1H, t, J 7.9 Hz); 7.49 (3H, d, J 8.1 Hz); 7.41 (1H, d, J 7.7 Hz); 4.67 (2H, mult); 4.16 (2H, q, J 7.2 Hz); 2.89 (2H, t, J 7.6 Hz); 2.62 (2H, t, J 7.6 Hz); 2.42 (1H, mult); 2.08 (3H, s); 1.33 (2H, mult); 1.28 (3H, t, J 7.2 Hz); 1.01 (2H, mult).

APCI-MS m/z: 591.6 [MH⁺].

### EXAMPLE 26

### 3-{5-({[4-(Cyclopropylsulfonyl)benzyl]amino}carbonyl)-2-methyl-6-oxo-1-[3-(trifluoromethyl)phenyl]-1,6-dihydropyridin-3-yl}propanoic acid

A mixture of ethyl 3-{5-({[4-(cyclopropylsulfonyl)benzyl]amino}carbonyl)-2-methyl-6-oxo-1-[3-(trifluoromethyl)phenyl]-1,6-dihydropyridin-3-yl}propanoate (Example 25, 108.8 mg, 0.18 mmol); methanol (1 ml), THF (1 ml), water (0.5 ml) and 2M sodium hydroxide solution (0.2 μl, 0.4 mmol) was stirred at ambient temperature for 20 min. The solution was acidified with acetic acid, diluted with water and purified by preparative HPLC to give the title compound as a white solid (88.1 mg, 87%).

¹H NMR (CDCl₃): δ 10.10 (1H, t, J 5.9 Hz); 8.58 (1H, s); 7.82 (2H, d, J 8.4 Hz); 7.80 (1H, s); 7.74 (1H, t, J 7.9 Hz); 7.49 (3H, d, J 8.1 Hz); 7.42 (1H, d, J 8.0 Hz); 4.67 (2H, mult); 2.91 (2H, t, J 7.4 Hz); 2.70 (2H, t, J 7.4 Hz); 2.42 (1H, mult); 2.09 (3H, s); 1.32 (2H, mult); 1.00 (2H, mult).

APCI-MS m/z: 563.6 [MH⁺].

### Human Neutrophil Elastase Quenched-FRET Assay

The assay uses Human Neutrophil Elastase (HNE) purified from serum (Calbiochem art. 324681; Ref. Baugh, R. J. et al., 1976, Biochemistry. 15, 836-841). H was stored in 50 mM sodium acetate (NaOAc), 200 mM sodium chloride (NaCl), pH 5.5 with added 30% glycerol at −20° C. The protease substrate used was Elastase Substrate V Fluorogenic, MeOSuc-AAPV-AMC (Calbiochem art. 324740; Ref. Castillo, M. J. et al., 1979, Anal. Biochem. 99, 53-64). The substrate was stored in dimethyl sulphoxide (DMSO) at −20° C. The assay additions were as follows: Test compounds and controls were added to black 96-well flat-bottom plates (Greiner 655076), 1 μL in 100% DMSO, followed by 30 μL HNE in assay buffer with 0.01% Triton (trade mark) X-100 detergent. The assay buffer constitution was: 100 mM Tris(hydroxymethyl)aminomethane (TRIS) (pH 7.5) and 500 mM NaCl. The enzyme and the compounds were incubated at room temperature for 15 minutes. Then 30 μl substrate in assay buffer was added. The assay was incubated for 30 minutes at room temperature. The concentrations of HNE enzyme and substrate during the incubation were 1.7 nM and 100 μM, respectively. The assay was then stopped by adding 60 μl stop solution (140 mM acetic acid, 200 mM sodium monochloroacetate, 60 mM sodium acetate, pH 4.3). Fluorescence was measured on a Wallac 1420 Victor 2 instrument at settings: Excitation 380 nm, Emission 460 nm. IC₅₀ values were determined using Xlfit curve fitting using model 205.

When tested in the above screen, the compounds of the Examples gave IC₅₀ values for inhibition of human neutrophil elastase activity of less than 30 μM (micromolar), indicating that the compounds of the invention are expected to possess useful therapeutic properties. Specimen results are shown in the following Table:

| | Inhibition of Human |
|---|---|
| | Neutrophil Elastase IC₅₀ |
| Compound of | (micromolar, μM) |
| Example 4 | 0.031 |
| Example 6 | 0.045 |
| Example 7 | 0.048 |
| Example 20 | 0.023 |
| Example 23 | 0.061 |

1. A compound of formula 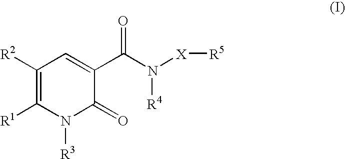
wherein
R¹ represents hydrogen or C₁-C₆ alkyl;
R² represents halogen, cyano, carboxyl, hydroxyl, nitro, —C(O)H, —C(O)NR¹⁰R¹¹, —NR¹²R¹³ or a group selected from C₁-C₆ alkyl, C₁-C₆ alkoxy, C₁-C₆ alkylcarbonyl, C₁-C₆ alkoxycarbonyl, C₂-C₆ alkenyl, C₂-C₆ alkynyl and a saturated or unsaturated 3- to 10-membered ring system optionally comprising at least one ring heteroatom selected from nitrogen, oxygen and sulphur, each group being optionally substituted by one or more substituents independently selected from halogen, cyano, carboxyl, hydroxyl, oxygen, nitro, —S(O)ₚR¹⁵, —NR¹⁶S(O)_{q}R¹⁷, —C(O)NR¹⁸R¹⁹, C₁-C₆ alkyl, C₁-C₆ alkoxy, C₁-C₆ alkylcarbonyl, C₁-C₆ alkoxycarbonyl and a saturated or unsaturated 5- to 6-membered monocyclic ring system optionally comprising at least one ring heteroatom selected from nitrogen, oxygen and sulphur;
R³ represents a phenyl group substituted with at least one substituent selected from halogen, cyano, nitro, trifluoromethyl or methylcarbonyl;
R⁴ represents hydrogen or C₁-C₆ alkyl optionally substituted with at least one substituent selected from hydroxyl and C₁-C₆ alkoxy;
X represents a bond or a group —C₁-C₆ alkylene-Y—, wherein Y represents a single bond, oxygen atom, NR²⁴ or S(O)_{w};
R⁵ represents a monocyclic ring system selected from
i) phenoxy,
ii) phenyl,
iii) a 5- or 6-membered heteroaromatic ring comprising at least one ring heteroatom selected from nitrogen, oxygen and sulphur,
iv) a saturated or partially unsaturated C₃-C₆ hydrocarbyl ring, or
v) a saturated or partially unsaturated 4- to 7-membered heterocyclic ring comprising at least one ring heteroatom selected from oxygen, S(O)ᵣ and NR²⁰, wherein at least one of the ring carbon atoms may be optionally replaced by a carbonyl group;
R⁵ being substituted by at least one substituent selected from oxygen, C₃-C₈ cycloalkyl, —S(O)ᵥR²¹, and C₁-C₆ alkyl substituted with at least one substituent selected from cyano, hydroxyl, C₁-C₆ alkoxy, C₁-C₆ alkylthio and —C(O)NR²²R²³;
R¹⁰, R¹¹, R¹² and R¹³ each independently represent hydrogen or C₁-C₆ alkyl;
p is 0, 1 or 2;
q is 0, 1 or 2;
r is 0, 1 or 2;
w is 0, 1 or 2;
R¹⁵, R¹⁶, R¹⁷, R¹⁸ and R¹⁹ each independently represent hydrogen or C₁-C₆ alkyl;
R²⁰ represents hydrogen, C₁-C₆ alkyl, C₁-C₆ alkylcarbonyl or C₁-C₆ alkoxycarbonyl;
v is 0, 1 or 2;
R²¹ represents hydrogen, C₁-C₆ alkyl or C₃-C₈ cycloalkyl;
R²² and R²³ each independently represent hydrogen or C₁-C₆ alkyl;
R²⁴ represents hydrogen or C₁-C₆ alkyl;
with the proviso that when R⁵ is substituted with a C₃-C₈ cycloalkyl or an —S(O)ᵥR²¹ substituent group, then R² represents either
(a) a substituted C₁-C₆ alkyl group in which at least one substituent group is cyano, carboxyl, —S(O)ₚR¹⁵, —NR¹⁶S(O)_{q}R¹⁷, —C(O)NR¹⁸R¹⁹ or C₁-C₆ alkoxycarbonyl
(b) a substituted C₂-C₆ alkynyl group in which at least one substituent group is hydroxyl, or
(c) a substituted C₁-C₆ alkoxy group in which at least one substituent group is a 5- to 6-membered saturated or unsaturated monocyclic ring system optionally comprising at least one ring heteroatom selected from nitrogen, oxygen and sulphur;
or a pharmaceutically acceptable salt thereof.

2. The compound according to claim 1, wherein R¹ represents C₁-C₆ alkyl.

3. The compound according to claim 1, wherein R² represents halogen, or a group selected from C₁-C₄ alkyl, C₁-C₄ alkoxy, C₂-C₄ alkynyl and a saturated or unsaturated 3- to 6-membered ring system optionally comprising two ring heteroatoms independently selected from nitrogen and oxygen, each group being optionally substituted by one or two substituents independently selected from cyano, carboxyl, hydroxyl, —S(O)ₚR¹⁵, —NR¹⁶S(O)_{q}R¹⁷, —C(O)NR¹⁸R¹⁹, C₁-C₄ alkyl, C₁-C₄ alkoxycarbonyl and a saturated or unsaturated 5- to 6-membered monocyclic ring system optionally comprising two ring heteroatoms independently selected from nitrogen and oxygen.

4. The compound according to claim 1, wherein R³ represents a phenyl group substituted with a trifluoromethyl substituent.

5. The compound according to claim 1, wherein R⁵ represents a monocyclic ring system selected from phenyl or a 5- or 6-membered heteroaromatic ring comprising one or two ring heteroatoms independently selected from nitrogen and oxygen, the monocyclic ring system being substituted by one or two substituents independently selected from C₃-C₆ cycloalkyl, —S(O)ᵥR²¹, and C₁-C₄ alkyl substituted with one or two substituents independently selected from cyano, hydroxyl, C₁-C₄ alkoxy, C₁-C₄ alkylthio and —C(O)NR²²R²³.

6. The compound of formula (I) as defined in claim 1 selected from:
N-{[3-(2-Hydroxyethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
5-(3,5-Dimethylisoxazol-4-yl)-N-{[3-(2-hydroxyethyl)isoxazol-5-yl]methyl}-6-methyl-2-oxo-1-[3-(trifluoromethyl)phenyl]-1,2-dihydropyridine-3-carboxamide;
N-{[3-(2-Hydroxyethyl)isoxazol-5-yl]methyl}-5-iodo-6-methyl-2-oxo-1-[3-(trifluoromethyl)phenyl]-1,2-dihydropyridine-3-carboxamide;
N-{[3-(Hydroxymethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
5-(3,5-Dimethylisoxazol-4-yl)-N-{[3-(hydroxymethyl)isoxazol-5-yl]methyl}-6-methyl-2-oxo-1-[3-(trifluoromethyl)phenyl]-1,2-dihydropyridine-3-carboxamide;
5-Ethyl-N-{[3-(hydroxymethyl)isoxazol-5-yl]methyl}-6-methyl-2-oxo-1-[3-(trifluoromethyl)phenyl]-1,2-dihydropyridine-3-carboxamide;
5-Cyclopropyl-N-{[3-(hydroxymethyl)isoxazol-5-yl]methyl}-6-methyl-2-oxo-1-[3-(trifluoromethyl)phenyl]-1,2-dihydropyridine-3-carboxamide:
N-{[3-(Methoxymethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
5-(3,5-Dimethylisoxazol-4-yl)-6-methyl-N-({3-[(methylthio)methyl]isoxazol-5-yl}methyl)-2-oxo-1-[3-(trifluoromethyl)phenyl]-1,2-dihydropyridine-3-carboxamide;
N-{[3-(3-Amino-3-oxopropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
N-{[3-(2-Cyanoethyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
N-{[3-(3-Hydroxypropyl)isoxazol-5-yl]methyl}-6-methyl-5-(1-methyl-1H-pyrazol-5-yl)-2-oxo-1-[3-(trifluoromethyl)phenyl]-1,2-dihydropyridine-3-carboxamide;
5-(3-Amino-3-oxopropyl)-N-[(3-cyclopropylisoxazol-5-yl)methyl]-6-methyl-2-oxo-1-[3-(trifluoromethyl)phenyl]-1,2-dihydropyridine-3-carboxamide;
5-(2-Cyanoethyl)-N-[(3-cyclopropylisoxazol-5-yl)methyl]-6-methyl-2-oxo-1-[3-(trifluoromethyl)phenyl]-1,2-dihydropyridine-3-carboxamide;
N-[(3-Cyclopropylisoxazol-5-yl)methyl]-5-[3-(dimethylamino)-3-oxopropyl]-6-methyl-2-oxo-1-[3-(trifluoromethyl)phenyl]-1,2-dihydropyridine-3-carboxamide;
3-{5-({[(3-Cyclopropylisoxazol-5-yl)methyl]amino}carbonyl)-2-methyl-6-oxo-1-[3-(trifluoromethyl)phenyl]-1,6-dihydropyridin-3-yl}propanoic acid;
N-[(3-Cyclopropylisoxazol-5-yl)methyl]-6-methyl-5-[3-(methylsulfonyl)propyl]-2-oxo-1-[3-(trifluoromethyl)phenyl]-1,2-dihydropyridine-3-carboxamide;
N-[(3-Cyclopropylisoxazol-5-yl)methyl]-6-methyl-5-{3-[(methylsulfonyl)amino]propyl}-2-oxo-1-[3-(trifluoromethyl)phenyl]-1,2-dihydropyridine-3-carboxamide;
6-Methyl-5-{3-[(methylsulfonyl)amino]propyl}-N-[4-(methylsulfonyl)benzyl]-2-oxo-1-[3-(trifluoromethyl)phenyl]-1,2-dihydropyridine-3-carboxamide;
5-(3-Hydroxyprop-1-yn-1-yl)-6-methyl-N-[4-(methylsulfonyl)benzyl]-2-oxo-1-[3-(trifluoromethyl)phenyl]-1,2-dihydropyridine-3-carboxamide;
5-(3-Amino-3-oxopropyl)-N-[4-(cyclopropylsulfonyl)benzyl]-6-methyl-2-oxo-1-[3-(trifluoromethyl)phenyl]-1,2-dihydropyridine-3-carboxamide;
N-[4-(Isopropylsulfonyl)benzyl]-6-methyl-5-(2-morpholin-4-ylethoxy)-2-oxo-1-[3-(trifluoromethyl)phenyl]-1,2-dihydropyridine-3-carboxamide;
N-[4-(Cyclopropylsulfonyl)benzyl]-6-methyl-5-[(methylsulfonyl)methyl]-2-oxo-1-[3-(trifluoromethyl)phenyl]-1,2-dihydropyridine-3-carboxamide;
5-(1-Cyanoethyl)-6-methyl-N-[4-(methylsulfonyl)benzyl]-2-oxo-1-[3-(trifluoromethyl)phenyl]-1,2-dihydropyridine-3-carboxamide;
Ethyl 3-{5-({[4-(cyclopropylsulfonyl)benzyl]amino}carbonyl)-2-methyl-6-oxo-1-[3-(trifluoromethyl)phenyl]-1,6-dihydropyridin-3-yl}propanoate; and
3-{5-({[4-(Cyclopropylsulfonyl)benzyl]amino}carbonyl)-2-methyl-6-oxo-1-[3-(trifluoromethyl)phenyl]-1,6-dihydropyridin-3-yl}propanoic acid;
and pharmaceutically acceptable salts thereof.

7. A process for the preparation of a compound of formula (I) or a pharmaceutically acceptable salt thereof as defined in claim 1 which comprises,
(a) reacting a compound of formula 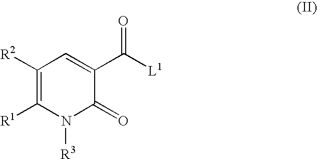
wherein L¹ represents a leaving group and R¹, R² and R³ are as defined in claim 1,
with a compound of formula 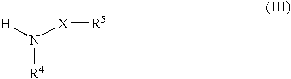
wherein X, R⁴ and R⁵ are as defined in claim 1; or
(b) when R² represents a halogen atom, reacting a compound of formula 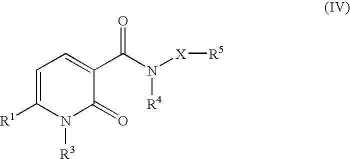
wherein X, R¹. R³, R⁴ and R⁵ are as defined in claim 1, with a halogenating agent; or
(c) when R² is other than a halogen atom, reacting a compound of formula 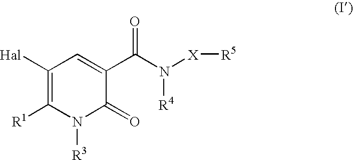
wherein Hal represents a halogen atom and X, R¹, R³, R⁴ and R⁵ are as defined in claim 1,
with a nucleophile R^{2′}-M wherein R^{2′} is R² as defined in claim 1 other than a halogen atom and M represents an organo-tin or organo boronic acid group;
and optionally after (a), (b) or (c) carrying out one or more of the following:
converting the compound obtained to a further compound according to claim 1
forming a pharmaceutically acceptable salt of the compound.

8. A pharmaceutical composition comprising a compound of formula (I) or a pharmaceutically acceptable salt thereof as claimed in claim 1 or claim 6 in association with a pharmaceutically acceptable adjuvant, diluent or carrier.

9. A process for the preparation of a pharmaceutical composition as claimed in claim 8 which comprises mixing a compound of formula (I) or a pharmaceutically acceptable salt thereof as claimed in claim 1 or claim 6 with a pharmaceutically acceptable adjuvant, diluent or carrier.

10-12. (canceled)

13. A method of treating, or reducing the risk of, a disease or condition in which inhibition of neutrophil elastase activity is beneficial which comprises administering to a patient in need thereof a therapeutically effective amount of a compound of formula (I) or a pharmaceutically acceptable salt thereof as claimed in claim 1.

14. A method of treating, or reducing the risk of, an inflammatory disease or condition which comprises administering to a patient in need thereof a therapeutically effective amount of a compound of formula (I) or a pharmaceutically acceptable salt thereof as claimed in claim 1.

15. The method according to claim 13 or claim 14, wherein the disease or condition is adult respiratory distress syndrome (ARDS), cystic fibrosis, pulmonary emphysema, bronchitis, bronchiectasis, chronic obstructive pulmonary disease (COPD), pulmonary hypertension, asthma, rhinitis, ischemia-reperfusion injury, rheumatoid arthritis, osteoarthritis, cancer, atherosclerosis or gastric mucosal injury.
